(19) United States
(12) Patent Application Publication
(10) Pub. No.: US 2026/0165921 A1
(43) Pub. Date: Jun. 18, 2026

(54) COMPOSITIONS AND METHODS FOR LIGHTENING HAIR

(71) Applicant: L'Oreal, Paris (FR)

(72) Inventors: Cho-cho KHINE, Scotch Plains, NJ (US); Tathagata MUKHERJEE, Union, NJ (US); Martin ASARE, Springfield, NJ (US); Ronak RUGHANI, Edison, NJ (US)

(21) Appl. No.: 18/986,272

(22) Filed: Dec. 18, 2024

(51) Int. Cl. A61K 8/23; A61K 8/25; A61K 8/36; A61K 8/73; A61Q 5/08

(52) U.S. Cl. CPC A61K 8/23; A61K 8/25; A61K 8/36; A61K 8/738; A61Q 5/08

(57) ABSTRACT

The disclosure relates to compositions for lightening the color of hair, methods of using the compositions, and kits comprising the compositions. The compositions can provide a very high level of lift while minimizing damage to the hair, and simultaneously provide frizz control, curl retention, and cosmetic benefits such as softness and smoothness to the treated hair.

### TECHNICAL FIELD

The present disclosure relates to compositions for lightening hair, kits comprising the compositions, and methods for using the compositions. In addition to lightening the hair, the compositions and methods provide additional benefits such as reduced damage, improved look and feel of the hair, frizz control, and shape retention.

### BACKGROUND

It is known that consumers desire to use cosmetic and care compositions that enhance the appearance of keratinous substrates such as hair, e.g., by changing the color of the hair, and/or by imparting various properties to hair, such as shine and conditioning. The process of altering the color of hair may, for example, involve lifting the color of the hair to lighten the hair color, and/or depositing an artificial color onto the hair which provides a different or darker shade or color to the hair. The process of lifting the color of hair, also known as lightening, generally requires the use of compositions that comprise oxidizing agents. Such oxidizing agents enter the hair shaft and react with melanin in the hair, thereby lightening the color of the hair.

Variation of tone height before and after the application of a lightening composition is used to evaluate lightening or lifting of the color of the hair. Degree or level of lightening or lift corresponds to the variation. The notion of “tone” is based on the classification of the natural shades, one tone separating each shade from the shade immediately following or preceding it, which is well known to hairstyling professionals. Tone heights or levels range from 1 (black) to 10 (light blonde) or even up to 12 (platinum), one unit corresponding to one tone. Thus, the higher the number, the lighter the shade or the greater the degree of lift.

Many consumers seek high degrees of lift, such as when going from black to light blonde. The level of lift desired is typically achieved by using varying amounts of oxidizing and/or alkaline agents in the lightening composition, or by varying the length of time the composition is left on the hair. In cases where high levels of lift are sought (e.g. at least 6), multiple applications of the lightening composition are often required to achieve the desired shade.

Additionally, consumers also often seek tones that are lighter and brighter. This can be achieved by altering the undertones of the hair color. Typically, undertones are altered or neutralized by applying a separate toning composition to the hair after the hair is lightened, for example to make the tone appear warmer or cooler, get rid of brassy undertones, etc.

However, compositions for lightening and toning hair are known to be harsh and to damage the hair fibers. Compositions and processes that are used for high (e.g. at least 6) levels of lift are particularly damaging due to the need for increased amounts of harsh chemicals to achieve the desired lightening effect and for increased processing times, meaning that the damaging compositions are in contact with the hair fibers for a longer period of time. In addition, sometimes it is necessary to perform the lightening process more than once, especially when high or very high levels of lift are desired. Such multiple processing applications are also particularly damaging to hair. All of these treatments therefore lead to hair that feels rough, tangles easily, is frizzy, and generally looks unhealthy. The damage can also lead to a loss in the curliness of hair, which causes consumers with curly hair to have to choose between maintaining their curls or lightening their hair. Thus, in order to reduce or avoid damage to the hair, as well as to improve the cosmetic performance of these compositions, new lightening compositions and/or processes for achieving the desired level of lift, preferably in less time, with fewer applications, with reduced amounts of harsh chemicals, and/or with additives that can minimize the damage are needed.

Lightening compositions typically comprise persulfates which accelerate the speed of the hair lightening process. On the one hand this is advantageous because the harsh chemical treatment can be removed from the hair after a relatively shorter period of time, thus permitting less exposure of the hair fibers to the damaging compositions. On the other hand, however, persulfates themselves are a contributing cause of hair damage. Moreover, the reduced processing time must also be balanced against the amount of time needed to achieve the desired level of lift, because if the composition is removed too soon, the active agents will not have had sufficient time to de-colorize the hair fibers and the desired level of lightening will not be achieved.

There have been various attempts to mitigate hair damage during the lightening process. For example, some bleaching compositions contain additional components intended to reduce the damage to the hair fiber. However, this approach poses difficulties insofar as some components cause the compositions to be unstable, are detrimental to cosmetic attributes of the treated hair, and/or present other problems such as lack ease and uniformity of application, poor rheology or viscosity of the compositions, and/or reduced efficacy in lifting or lightening the color of the hair. Other compositions have been modified to remove certain damaging components, but these compositions are not able to achieve the desired level of lightening in the time the compositions can be left on the hair before damage occurs. It would, therefore, be desirable to provide compositions and methods that can lift the color of hair in an effective and expeditious manner, while leaving the hair with satisfactory cosmetic properties.

It has now been surprisingly discovered that hair lightening compositions according to the disclosure having certain beneficial additives and reduced amounts of certain harsh chemicals such as persulfate compounds and oxidizing agents can be prepared which permit effective and efficient lightening, while also permitting curl retention and providing a smooth feel and healthy appearance of the treated hair.

### SUMMARY

The present disclosure relates to compositions and methods for lightening or lifting the color of the hair, which are able to achieve high or very high levels of lift, even with lower amounts of persulfates and oxidizing agents, while also providing additional benefits such as smoothness and frizz control to the treated hair, and curl retention to curly hair. Damage during the lightening/bleaching process is minimized, which protects the integrity of the hair and leaves the hair stronger, softer, and with a healthier appearance than hair lightened with traditional hair bleaching compositions.

The disclosure relates to a three-part system comprising (1) a base lightening composition which comprises reduced amounts of harsh chemicals such as persulfate compounds, (2) an additive composition, and (3) an oxidizing composition or oxidizing agent, which can be mixed together at or near the time of use to produce the hair lightening compositions according to the disclosure.

The (1) base lightening compositions comprise (a) at least one persulfate compound, for example at least two persulfate compounds, and (b) at least one silicate compound. The total amount of (a) persulfate compounds may vary, but is advantageously less than about 50%, such as less than about 45%, or less than about 40%, for example may range from about 10% to not greater than 50% or from about 25% to about 45% by weight, relative to the total weight of the base lightening composition. In some embodiments, the base lightening compositions comprise (a) (i) ammonium persulfate and (a) (ii) at least one additional persulfate compound such as potassium persulfate and/or sodium persulfate. It has been discovered that choosing amounts of persulfate compounds relative to each other, high or very high levels of lift can be achieved in the same or even less time than with traditional base lightening compositions, leading to reduced damage to the hair. For example, in various embodiments the base lightening compositions may comprise ammonium persulfate and at least one additional persulfate compound, where the weight ratio of the amount of (a) (i) ammonium persulfate to the total amount of (a) (ii) additional persulfate compounds is equal to or less than about 1, for example ranges from about 0.1 to about 0.9, such as from about 0.2 to about 0.8, from about 0.3 to about 0.7, or from about 0.4 to about 0.6.

The total amount of (b) silicate compounds may vary, but generally ranges from about 5% to about 50%, such as from about 15% to about 45%, or from about 25% to about 40% by weight, based on the total weight of the base lightening composition. Optionally the base lightening compositions comprise at least one metasilicate, for example sodium metasilicate. In some embodiments, the base lightening compositions comprise at least two silicate compounds where at least one is a metasilicate, for example sodium silicate and sodium metasilicate.

The base lightening compositions may further comprise additional components such as alkalizing agents, thermal control agents, oxidizing agents, organic acids and their salts, oils, fillers, thickening agents, anionic surfactants, amino acids and their salts, and auxiliary components such as preservatives, hair conditioning agents, vitamins, chelating agents, pigments or colorants to color the composition, or any other component typically used in hair color lightening base compositions. Useful alkalizing agents include both organic and mineral alkalizing agents. It may in various embodiments be advantageous to include organic acids and their salts and/or amino acids and their salts to increase the integrity of the treated hair fibers. Useful thermal control agents may include urea, sodium stearate, and/or mineral oil. Thermal control agents that can be used may, in various embodiments, permit the hair lightening composition to achieve a peak temperature that is less than 70° C., for example less than about 65° C., less than about 63° C., less than about 60° C., less than about 58° C., less than about 55° C., less than about 53° C., or less than about 50° C.

The (2) additive compositions comprise (a) at least one carboxylic acid or salt thereof; (b) at least one cyclodextrin or derivative thereof; (c) at least one polysaccharide thickening agent; (d) at least one polyol; and (e) water. The pH of the additive composition is generally less than about 7. In various embodiments, the mole ratio of the (a) carboxylic acid(s) and salt(s) thereof to the (b) cyclodextrin(s) and derivative(s) thereof in the additive composition may range from about 0.5:1 to about 25:3, such as from about 4:1 to about 15:1, from about 5:1 to about 12:1, or from about 6:1 to about 10:1, or may be about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits. In some embodiments, the weight ratio of the (a) carboxylic acid(s) and salt(s) thereof to the (b) cyclodextrin(s) and derivative(s) thereof in the additive composition may range from about 1:3 to about 6:1, for example may be about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges using any of the foregoing as upper or lower limits. In various embodiments, the pH of the additive composition ranges from about 2 to about 6, such as from about 3 to about 5, from about 3.5 to about 5, from about 3 to about 4.5, from about 3.5 to about 4.5, or from about 3 to about 4.

In various embodiments, the (a) at least one carboxylic acid or salt thereof in the additive composition may be chosen from mono-carboxylic acids, di-carboxylic acids, tri-carboxylic acids, salts thereof, or mixtures thereof, such as, for example citric acid, lactic acid, malic acid, maleic acid, tartaric acid, glycolic acid, salts thereof, or mixtures thereof. In some further embodiments, suitable carboxylic acid(s) or salt(s) thereof may have a molecular weight of less than about 900 g/mol, preferably less than about 700 g/mol, less than about 500 g/mol, less than about 300 g/mol, or less than about 200 g/mol. In various embodiments, the total amount of the (a) carboxylic acids and salts thereof in the additive composition may range from about 0.1% to about 20%, preferably from about 0.1% to about 15%, from about 0.5% to about 15%, from about 0.5% to about 10%, from about 0.5% to about 5%, from about 1% to about 10%, or from about 1% to about 5% by weight, relative to the total weight of the additive composition.

In various embodiments, the (b) at least one cyclodextrin or derivative thereof in the additive composition may be chosen from compounds of formula:

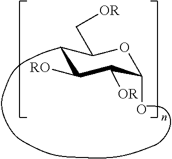

wherein R is chosen from H, CH₃, or a hydroxypropyl group, and n ranges from 6 to 8. For example, the at least one cyclodextrin or derivative thereof may be chosen from α-cyclodextrin, ß-cyclodextrin, γ-cyclodextrin, methyl-α-cyclodextrin, methyl-ß-cyclodextrin, methyl-γ-cyclodextrin, or mixtures thereof. The total amount of the (b) cyclodextrins and derivatives thereof may, in various embodiments, be present in an amount ranging from about 0.01% to about 20% by weight, such as, for example, from about 0.1% to about 18%, from about 0.5% to about 15%, from about 0.5% to about 10%, from about 0.5% to about 8%, from about 1% to about 13%, from about 1% to about 7%, or from about 1% to about 5% by weight, relative to the total weight of the additive composition.

In various embodiments, the (c) at least one polysaccharide thickening agent in the additive composition may be chosen from gums, for example gum arabic, tragacanth gum, karaya gum, guar gum, gellan gum, tara gum, locust bean gum, tamarind gum, xanthan gum, dehydroxanthan gum, Seneca gum, sclerotium gum, gellan gum, or the like. The total amount of polysaccharide thickening agent(s) may range from 0.05% to about 5%, preferably from about 0.1% to about 5%, more preferably from about 0.1% to about 3% by weight, relative to the total weight of the additive composition.

In various embodiments, the (d) at least one polyol in the additive composition may be chosen from ethylene glycol, propylene glycol, butylene glycol, hexylene glycol, pentylene glycol, diethylene glycol, dipropylene glycol, 1,3 propanediol, glycerin, polyethylene glycols, or combinations thereof. The total amount of (d) polyols may be at least 0.5%, for example may range from about 1% to about 10% by weight, relative to the total weight of the additive composition.

In various embodiments, water is present in the additive composition in an amount greater than 50%, for example ranging from about 70% to about 98%, from about 75% to about 98%, from about 80% to 98%, from about 85% to 98%, from about 90% to about 98%, from about 95% to about 98%, from about 70% to about 95%, from about 75% to about 95%, from about 80% to 95%, from about 85% to 95% by weight, relative to the total weight of the additive composition.

The (3) oxidizing composition (developer) or oxidizing agent that can be mixed with the base lightening composition or mixture to prepare the hair lightening composition at or near the time of use is not limited, and any conventional oxidizing composition or oxidizing agent for lightening hair can be used. Typically an oxidizing composition comprises an oxidizing agent such as peroxide, inorganic perhydrate salts, or a source of peroxymonocarbonate ions, in a cosmetically acceptable carrier. For example, the developer chosen as a solvent for preparing the base lightening mixture may be a 10V, 20V, 30V, or 40V aqueous hydrogen peroxide composition.

In further embodiments the disclosure relates to methods of lightening the color of hair using hair lightening compositions according to the disclosure.

In still further embodiments, the disclosure relates to methods of preparing hair lightening compositions comprising mixing or combining (1) a base lightening composition, (2) an additive composition, and (3) an oxidizing composition or oxidizing agent, as well as to hair lightening compositions prepared accordingly. For example, the base lightening composition may be mixed with the additive composition to form a base lightening mixture, and subsequently the base lightening mixture and oxidizing composition may be mixed, for example at or near the time of use. In various embodiments, the amount of additive composition that can be mixed with or added to the base lightening composition may range from about 1% to about 25%, such as from about 5% to about 20% by weight, relative to the total weight of the base lightening composition. In some embodiments, the additive composition is added to the base lightening composition, or mixed together with the base composition and oxidizing agent, so that a weight ratio of total persulfates in the base lightening composition to the amount of the additive composition added ranges from about 1:1 to about 5:1, such as from about 1:1 to about 4:1, or from about 1.5:1 to about 3.5:1. In still other embodiments, the amount of additive composition that can be mixed with or added to the base lightening composition may be chosen to provide a weight ratio of the total weight of the base lightening composition to the total weight of the additive composition ranging from about 5:1 to about 15:1, such as from about 6:1 to about 14:1, or about 7:1 to about 13.5:1.

In further embodiments still, the disclosure relates to kits comprising at least two, for example at least three, compartments or containers, wherein a first compartment or container comprises a base lightening composition according to the disclosure and a second compartment or container comprises an additive composition according to the disclosure. Additional compartment(s) or container(s) may, for example, comprise an oxidizing composition or oxidizing agent, a solvent for mixing with the base lightening composition to prepare the base lightening mixture, an implement to mix the base composition and an oxidizing composition or oxidizing agent and/or to apply the mixture to the hair, or one or more additional hair care compositions, for example comprising a pre- and/or post-treatment hair conditioning composition.

### BRIEF DESCRIPTION OF THE FIGURES

FIG. 1 is a graph showing the elastic modulus of hair treated with hair lightening compositions according to the disclosure (S9 and S12) and hair treated with comparative hair lightening compositions (S10 and S11).

FIG. 2 is a graph showing the cycles to break of hair treated with hair lightening compositions according to the disclosure (S9 and S12) and hair treated with comparative hair lightening compositions (S10 and S11).

FIG. 3 shows SEM images of hair treated with hair lightening compositions according to the disclosure (S9 and S12) and hair treated with comparative hair lightening compositions (S10 and S11).

FIG. 4 shows images of untreated curly hair (Control (T₂₄)), curly hair treated with hair lightening compositions according to the disclosure (S9 (T₂₄) and S12 (T₂₄)), and curly hair treated with comparative hair lightening compositions (S10 (T₂₄) and S11 (T₂₄)), all after 24 hours in high humidity conditions, compared to untreated before exposure to high humidity conditions (Control (T₀)).

FIG. 5 shows images of straight hair treated with a hair lightening composition according to the disclosure (S13) and straight hair treated with a comparative hair lightening composition (S14).

FIG. 6 shows photographs of a swatch of curly hair treated with a hair lightening composition according to the disclosure (S1) and a swatch of curly hair treated with a comparative hair lightening composition (S2).

FIGS. 7A-7B show photographs of swatches of curly hair treated with hair lightening compositions according to the disclosure (S3-S5) and swatches of curly hair treated with comparative hair lightening compositions (S6-S8).

### DETAILED DESCRIPTION

The disclosure relates to compositions that are useful for lightening or lifting the color of the hair, methods of lightening or lifting the color of the hair using the compositions, and kits that can be used in the methods.

### I. Compositions

It has been surprisingly and unexpectedly discovered that hair lightening compositions according to the disclosure effectively lighten or lift the color of the hair while minimizing the damaging effects of bleaching and/or of oxidizing agents in the compositions. Hair that is treated with the hair lightening compositions has less damage, looks and feels softer and smoother, and has less frizz than hair treated with conventional hair lightening compositions.

Hair lightening compositions according to the disclosure may, for example, be prepared by mixing (1) a base lightening composition according to the disclosure with (2) an additive composition to form a base lightening mixture, and combining the base lightening mixture with (3) an oxidizing composition or oxidizing agent to form a hair lightening composition. However, it should be understood that the order of mixing is not limited.

### Base Lightening Compositions

Base lightening compositions according to the disclosure include (a) at least one persulfate compound, for example at least two persulfate compounds, (b) one or more silicate compounds, and (c) optionally one or more compounds chosen from alkalizing agents other than persulfates, oxidizing agents, thermal control agents, organic acids and their salts, amino acids, amino sulfonic acids, oils, anionic surfactants, fillers, thickeners, or combinations of two or more thereof.

The base lightening compositions may be in various forms, including but not limited to powder, cream, lotion, or gel. In at least some embodiments, the base lightening compositions are anhydrous or substantially anhydrous, for example comprise less than 3%, such as less than 2%, less than 1.5%, less than 1%, less than 0.5%, less than 0.1%, or less than 0.01% water by weight, relative to the total weight of the base lightening composition. In some embodiments, the base lightening compositions are substantially or entirely pulverulent.

### Persulfate Compounds

Base lightening compositions according to the disclosure comprise at least one persulfate compound, for example at least two persulfate compounds. In a particular embodiment, the base lightening compositions comprise (i) ammonium persulfate, and (ii) at least one additional persulfate compound, for example potassium persulfate and/or sodium persulfate.

The total amount of persulfate compounds in base lightening compositions according to the disclosure is less than found in typical base lightening compositions, but surprisingly hair lightening compositions according to the disclosure are just as effective, and in some cases more effective, at lifting or lightening the color of the hair, even when high or very high levels of lift are desired. In various embodiments, the base lightening compositions will comprise less than about 50% persulfate compounds, such as no more than 50%, no more than 49%, no more than 48%, no more than 47%, no more than 46%, or no more than 45% persulfate compounds by weight, relative to the total weight of the base lightening composition. For example, base lightening compositions according to the disclosure typically comprise a total amount of persulfate compounds ranging from at least about 10%, such as at least about 15%, at least about 20%, at least about 25%, at least about 30%, at least about 35%, or at least about 40%, but less than about 50%, or less than about 45% by weight, relative to the total weight of the base lightening composition, including all ranges and subranges thereof. By way of non-limiting example, the total amount of persulfate compounds in the base lightening compositions may range from about 10% to about 50%, about 10% to about 45%, about 10% to about 40%, about 10% to about 35%, about 10% to about 30%, about 10% to about 25%, about 15% to about 50%, about 15% to about 45%, about 15% to about 40%, about 15% to about 35%, about 15% to about 30%, about 15% to about 25%, about 20% to about 50%, about 20% to about 45%, about 20% to about 40%, about 20% to about 35%, about 20% to about 30%, about 20% to about 25%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, or about 35% to about 40% by weight, relative to the total weight of the base lightening composition. As further examples, the total amount of persulfates may range from about 30% to about 40%, such as from about 31% to about 40%, from about 32% to about 40%, from about 33% to about 40%, about 30% to about 39%, from about 31% to about 39%, from about 32% to about 39%, from about 33% to about 38%, about 30% to about 38%, from about 31% to about 38%, from about 32% to about 38%, from about 33% to about 38%, about 30% to about 37%, from about 31% to about 37%, from about 32% to about 37%, or from about 33% to about 37% by weight, relative to the total weight of the base lightening composition. In some embodiments, the total amount of persulfates may be about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% by weight, relative to the total weight of the base lightening composition, including all ranges and subranges using any of the foregoing as upper and lower limits.

In embodiments where the base lightening composition comprises (i) ammonium persulfate and (ii) at least one additional persulfate compound, the total amount of (i) ammonium persulfate in the base lightening composition can range from at least about 5%, such as at least about 6%, at least about 7%, at least about 8%, at least about 9%, at least about 10%, but less than about 20%, less than about 19%, less than about 18%, less than about 17%, less than about 16%, or less than about 15% by weight, relative to the total weight of the base lightening composition, including all ranges and subranges thereof. In some embodiments, the total amount of (i) ammonium persulfate may be about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the base lightening composition, including all ranges and subranges using any of the foregoing as upper and lower limits.

In embodiments where the base lightening composition comprises (i) ammonium persulfate and (ii) at least one additional persulfate compound, the total amount of (ii) additional persulfate compounds in the base lightening composition disclosure can range from at least about 15%, such as at least about 16%, at least about 17%, at least about 18%, at least about 19%, at least about 20%, at least about 22%, at least about 23%, at least about 24%, or at least about 25%, but less than about 40%, less than about 35%, or less than about 30% by weight, relative to the total weight of the base lightening composition, including all ranges and subranges thereof. In some embodiments, the total amount of (ii) additional persulfate compounds may be about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight, relative to the total weight of the base lightening composition, including all ranges and subranges using any of the foregoing as upper and lower limits.

In various embodiments, it may be particularly advantageous to choose an amount of (i) ammonium persulfate that is equal to or less than the total amount of (ii) additional persulfate compounds, for example less than the total amount of potassium persulfate and/or sodium persulfate. As non-limiting examples, the base lightening compositions may comprise ammonium persulfate and at least one additional persulfate compound chosen from potassium persulfate and/or sodium persulfate, where the weight ratio of (i) the amount of ammonium persulfate to (ii) the total amount of additional persulfate compounds, for example potassium sulfate, or the weight ratio of the amount of ammonium persulfate to the total amount of additional persulfate compounds, for example potassium persulfate and sodium persulfate, is equal to or less than about 1. By way of example, the weight ratio may range from about 0.1 to about 0.9, such as from about 0.2 to about 0.8, from about 0.3 to about 0.7, or from about 0.4 to about 0.6. In some embodiments, the weight ratio of (i) the amount of ammonium persulfate to (ii) the total amount of additional persulfate compounds, for example potassium persulfate and/or sodium persulfate, is about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, or about 0.9, or may be any range using any of the foregoing as upper and lower limits.

### Silicate Compounds

Base lightening compositions according to the disclosure comprise at least one silicate compound. Non-limiting examples of silicates include lithium, sodium, and potassium silicates, metasilicates, and disilicates, and combinations of two or more thereof, including mixed lithium, sodium, and potassium salts thereof. Specific non-limiting examples include aluminum silicate, magnesium silicate, aluminum magnesium silicate, calcium silicate, barium silicate, strontium silicate, potassium silicate, potassium metasilicate, sodium silicate, sodium metasilicate, or any combination of two or more thereof. In at least some embodiments, the base lightening compositions comprise at least one metasilicate compound, for example sodium metasilicate. In some embodiments the base lightening compositions comprise at least two silicate compounds, wherein at least one is a metasilicate.

The total amount of silicate compounds in base lightening compositions according to the disclosure may, in at least some embodiments, be greater than found in typical base lightening compositions. In particular, it may be advantageous in some embodiments to increase the amount of silicate compounds in base lightening compositions according to the disclosure achieve and/or maintain a desired pH of the hair lightening compositions.

For example, the total amount of silicate compounds may range from about 5% to about 50%, such as from about 15% to about 45%, or from about 30% to about 40% by weight, based on the total weight of the base lightening composition. In various embodiments, the total amount of silicate compounds may range from about 5% to about 45%, about 5% to about 40%, about 5% to about 35%, about 5% to about 20%, about 10% to about 50%, about 10% to about 45%, about 10% to about 40%, about 10% to about 35%, about 10% to about 30%, about 15% to about 50%, about 15% to about 45%, about 15% to about 40%, about 15% to about 35%, about 15% to about 30%, about 20% to about 50%, about 20% to about 45%, about 20% to about 40%, about 20% to about 35%, about 20% to about 30%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, or about 35% to about 40% by weight, based on the total weight of the base lightening composition.

### Alkalizing Agents

The base lightening compositions according to the disclosure may optionally further comprise at least one alkalizing agent other than silicates. The alkalizing agents may be chosen from organic and/or mineral alkalizing agents, for example, organic amines and their organic or mineral salts, ammonium salts, and mineral alkalizing agents such as inorganic bases and hydroxide base compounds.

In various embodiments, it may be advantageous to include amounts of alkalizing agents in the base lightening compositions achieve and/or maintain a desired pH of the hair lightening compositions.

### Organic Alkalizing Agents

As non-limiting examples, suitable organic amines may be chosen from alkanolamines and/or derivatives of alkanolamines. In various embodiments, alkanolamines and/or derivatives of alkanolamines may be chosen from mono-, di- or trialkylamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinine, morpholine, pyridine, piperidine, pyrimidine, piperazine, triazine and derivatives thereof. In some embodiments, alkanolamines and/or derivatives of alkanolamines that may be mentioned include but not limited to: monoethanolamine (also known as ethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino)methane.

In one embodiment, the at least one alkalizing agent comprises, consists essentially of, or consists of alkanolamines such as monoethanolamine. In one embodiment, the alkalizing agent excludes alkanolamines, but may include derivatives of alkanolamines.

Another example of organic amines that can be used include compounds of formula (I):

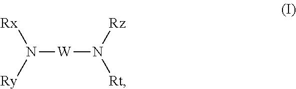

wherein:
W is chosen from C1-C6 alkylene residues optionally substituted with a hydroxyl group or a C1-C6 alkyl radical; and
Rx, Ry, Rz, and Rt, which may be identical or different, are chosen from a hydrogen atom, C1-C6 alkyl radicals, C1-C6 hydroxyalkyl radicals, and C1-C6 aminoalkyl radicals.

As non-limiting examples, compounds of formula (I) may be chosen from 1,3-diaminopropane, 1,3-diamino-2-propanol, spermine, and spermidine.

Useful organic salts include, by way of example only, citrates, lactates, glycolates, gluconates, acetates, propionates, fumarates, oxalates, and tartrates. Useful mineral salts include, by way of example only, hydrohalides (for example hydrochlorides), carbonates, hydrogen carbonates, sulfates, hydrogen phosphates, and phosphates.

Non-limiting examples of ammonium salts that may be used include carbonate and bicarbonate.

If present, the total amount of organic alkalizing agents may independently range from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4% by weight, based on the total weight of the base lightening composition.

### Mineral Alkalizing Agents

Useful and non-limiting mineral alkalizing agents include metal hydroxides and carbonates. Suitable metal hydroxides and carbonates include alkali metal and alkaline earth metal hydroxides or carbonates. Examples of such metal hydroxides include sodium, potassium, lithium, calcium, magnesium. In some embodiments, the alkalizing agent comprises ammonia, ammonium carbonates, sodium carbonates, potassium carbonates, ammonium bicarbonates, sodium bicarbonates, potassium bicarbonates, ammonium hydroxides, sodium hydroxides, potassium hydroxides, salts thereof, hydrates thereof, or mixtures thereof. In some embodiments, potassium carbonate may be used.

If present, the total amount of mineral alkalizing agents may range from about 0.1% to about 10% by weight, relative to the total weight of the base lightening composition, including all subranges therebetween. For example, the base lightening composition may contain from about 0.5% to about 10%, from about 0.75% to about 9%, from about 1% to about 8.5%, from about 1.5% to about 8%, from about 1.75% to about 7.5%, from about 2% to about 7%, from about 2.25% to about 6.5%, from about 2.5% to about 6%, from about 2.75% to about 5.5%, from about 3% to about 5.25%, or from about 3% to about 5% by weight, based on the total weight of the base lightening composition, including all ranges using any of the foregoing as upper and lower limits.

### Thermal Control Agents

It is known that when a base lightening composition is mixed with an aqueous developer to form a hair lightening composition, a chemical reaction occurs that raises the temperature of the mixture, for example to temperatures greater than about 70° C. (i.e. greater than about 160° F.), which can result in conditions that are uncomfortable for the user and also potentially unsafe. Therefore, in various embodiments the base lightening composition may optionally include one or more thermal control agents, for example sodium stearate, urea, mineral oil, magnesium carbonate, and/or magnesium carbonate hydroxide.

If present, the total amount of thermal control agents may vary, but typically ranges from about 1% to about 20%, such as from about 3% to about 18%, from about 5% to about 15%, or from about 8% to about 12%, or may be about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the base lightening composition, including all ranges and subranges using any of the foregoing as upper and lower limits.

For example, the total amount of urea may range from about 0.1% to about 15%, such as from about 0.5% to about 10%, from about 0.5% to about 8%, from about 0.5% to about 6%, from about 0.5% to about 5%, from about 0.5% to about 4%, from about 0.5% to about 3%, from about 0.5% to about 2%, from about 1% to about 10%, from about 1% to about 8%, from about 1% to about 6%, from about 1% to about 5%, from about 1% to about 4%, from about 1% to about 3%, from about 1% to about 2%, from about 1.5% to about 15%, from about 1.5% to about 10%, from about 1.5% to about 8%, from about 1.5% to about 6%, from about 1.5% to about 5%, from about 1.5% to about 4%, from about 1.5% to about 3%, from about 2% to about 10%, from about 2% to about 8%, from about 2% to about 6%, from about 2% to about 5%, from about 2% to about 4%, from about 2% to about 3%, from about 2.5% to about 10%, from about 2.5% to about 8%, from about 2.5% to about 6%, from about 2.5% to about 5%, from about 2.5% to about 4%, from about 2.5% to about 3%, from about 3% to about 10%, from about 3% to about 8%, from about 3% to about 6%, from about 3.5% to about 5%, from about 3.5% to about 4%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the base lightening composition, including all ranges and subranges using any of the foregoing as upper and lower limits.

As a further example, the base lightening compositions may comprise from about 0.1% to about 10%, such as from about 0.5% to about 7.5%, from about 1% to about 5%, or from about 1.5% to about 3.5% sodium stearate by weight, relative to the total weight of the base lightening composition. In some embodiments sodium stearate and mineral oil can be used together to work synergistically to control the temperature of the lightening composition without negatively impacting the level of lightening achieved. This surprising effect is most prominently seen when the weight ratio of sodium stearate to mineral oil ranges from about 1:3 to about 3:1, such as from about 1:2.5 to about 2.5:1, from about 1:2 to about 2:1, from about 1:1.5 to about 1.5:1, from about 1:1.25 to about 1.25:1, or from about 1:1.1 to about 1.1:1, or is about 1:1. If present, the amounts of sodium stearate and mineral oil may vary, but may, for example, independently range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, or about 5% by weight, relative to the total weight of the base lightening composition, including all ranges and subranges using any of the foregoing as upper and lower limits.

### Oxidizing Agents

Base lightening compositions according to the disclosure optionally comprise at least one oxidizing agent other than persulfates. Useful and non-limiting examples of oxidizing agents include perborates, percarbonates, peroxides, organic peracids, salts thereof, or mixtures thereof. In some embodiments, the base lightening compositions can include, as oxidizing agents, one or more of alkali metal bromates, ferricyanides, redox enzymes such as laccases, peroxidases, solid hydrogen peroxides such as urea hydrogen peroxide, sodium peroxide, and strontium peroxide, and 2-electron oxidoreductases, such as uricase.

If present, the total amount of oxidizing agents other than persulfates in the base lightening compositions may, in various embodiments, range from about 0.5% to about 45%, such as from about 1% to about 40%, from about 1.5% to about 35%, from about 2% to about 30%, from about 2.5% to about 25%, from about 3% to about 20%, from about 3.5% to about 15%, from about 4% to about 12%, from about 4.5% to about 10%, or from about 5% to about 8% by weight, based on the total weight of the hair base lightening composition, including all ranges using any of the foregoing as upper and lower limits. In at least some embodiments, the base lightening composition is free or substantially free of oxidizing agents other than persulfates.

### Organic Acids and Salts of Organic Acids

Optionally, base lightening compositions according to the disclosure comprise at least one organic acid, for example chosen from carboxylic acids. It is believed that the use of organic acids provides advantageous bonding benefits to the hair. Salts of organic acids may also be chosen, and are expressly included in the terms “organic acid” and “carboxylic acid” whether or not so stated, unless expressly indicated otherwise.

In various embodiments, the organic acids may be chosen from non-polymeric mono-, di-, and/or tri-carboxylic acids. Non-limiting examples of useful carboxylic acids include citric acid, maleic acid, succinic acid, aspartic acid, glutamic acid, lactic acid, malic acid, tartaric acid, glutaric acid, acetic acid, glycolic acid, oxalic acid, salts thereof, or combinations thereof. Derivatives of the foregoing, e.g. keto or oxo derivatives, may also be chosen. In some embodiments, the organic acid chosen comprises, consists essentially of, or consists of citric acid and salts thereof, such as sodium citrate. In at least some preferred embodiments, sodium citrate is used.

If present, the total amount of organic acids and salts thereof may range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, or about 7% by weight, relative to the total weight of the base lightening composition, including all ranges and subranges using any of the foregoing as upper and lower limits.

### Amino Acids

Optionally, the base lightening compositions comprise one or more amino acids. It is believed that the use of amino acids provides advantageous bonding benefits to the hair, particularly in combination with organic acids and salts. Salts of amino acids may also be chosen, and are expressly included in the term “amino acid” whether or not so stated. As used herein, the term “amino acid” includes amino carboxylic acids and salts thereof as well as amino sulfonic acids and salts thereof. As used herein, amino acids are understood to refer to organic compounds containing a carboxylic acid group (—COOH) (amino carboxylic acids) and/or sulfonic acid group (—S(═O)₂—OH) (amino sulfonic acids) and an amino group (—NH₂) which may be primary or secondary, or may be intra-cyclic, along with a side chain (R group) specific to each amino acid.

Non-limiting examples of amino carboxylic acids that may be chosen include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and combinations of two or more thereof. Exemplary amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoluene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, and combinations of two or more thereof.

By way of example, in some embodiments the base lightening compositions comprise one or more amino carboxylic acids of formula (II):

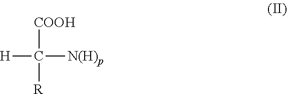

wherein:
p is an integer equal to 1 or 2, and
when p=1, R forms, together with the nitrogen atom, a saturated heterocycle comprising from 5 to 8 ring members, preferably 5 ring members, it being possible for this ring to be substituted by one or more groups chosen from hydroxyl or (C1-C4)alkyl; or
when p=2, R represents a hydrogen atom or a saturated, linear, or branched (C1-C12)alkyl group, preferably a (C1-C4)alkyl group, optionally interrupted by one or more heteroatoms or groups chosen from —S—, —NH—, or —C(NH)—, and/or optionally substituted by one or more groups chosen from hydroxyl (—OH), amino (—NH₂), —SH, —COOH, —CONH2, —NH—C(NH)—NH2, or an imidazole ring.

In some embodiments, the base lightening compositions comprise one or more amino acids chosen from arginine, glycine, proline, methionine, serine, lysine, histidine, salts of any of the foregoing (e.g. alkali metal, alkaline earth metal, or zinc salts), or combinations of two or more thereof. In at least certain embodiments, the amino acid compounds in the base lightening composition consist essentially of or consist of amino acids chosen from glycine or salts thereof.

In various embodiments, the total amount of amino acids may range from about 0.1% to about 10%, such as from about 0.5% to about 10%, from about 0.5% to about 7.5%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.5%, from about 1% to about 10%, from about 1% to about 7.5%, from about 1% to about 5%, from about 1% to about 4.5%, from about 1% to about 4%, from about 1% to about 3.5%, from about 1% to about 3%, from about 1% to about 2.5%, from about 1% to about 2%, from about 1% to about 1.5%, from about 1.25% to about 10%, from about 1.25% to about 7.5%, from about 1.25% to about 5%, from about 1.25% to about 4.5%, from about 1.25% to about 4%, from about 1.25% to about 3.5%, from about 1.25% to about 3%, from about 1.25% to about 2.5%, from about 1.25% to about 2%, from about 1.25% to about 1.5%, from about 1.5% to about 10%, from about 1.5% to about 7.5%, from about 1.5% to about 5%, from about 1.5% to about 4.5%, from about 1.5% to about 4%, from about 1.5% to about 3.5%, from about 1.5% to about 3%, from about 1.5% to about 2.5%, from about 1.5% to about 2%, from about 1% to about 1.5%, from about 1.75% to about 10%, from about 1.75% to about 7.5%, from about 1.75% to about 5%, from about 1.75% to about 4.5%, from about 1.75% to about 4%, from about 1.75% to about 3.5%, from about 1.75% to about 3%, from about 1.75% to about 2.5%, from about 1.75% to about 2%, from about 2% to about 10%, from about 2% to about 7.5%, from about 2% to about 5%, from about 2% to about 4.5%, from about 2% to about 4%, from about 2% to about 3.5%, from about 2% to about 3%, from about 2% to about 2.5%, from about 2.25% to about 10%, from about 2.25% to about 7.5%, from about 2.25% to about 5%, from about 2.25% to about 4.5%, from about 2.25% to about 4%, from about 2.25% to about 3.5%, from about 2.25% to about 3%, from about 2.25% to about 2.5%, or may be about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, or about 5% by weight, relative to the total weight of the base lightening composition, including all ranges and subranges using any of the foregoing as upper and lower limits.

### Oils

In some cases, the base lightening compositions include one or more oils. Non-limiting examples of oils include ester oils, silicone oils, fluoro oils, vegetable oils, animal oils, hydrocarbon-based oils, or mixtures thereof. For example, lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, shea butter, or combinations of two or more thereof can be used. Mention is made, as an example of linear or branched hydrocarbons, of oils of mineral or synthetic origin, such as liquid paraffins and their derivatives, petrolatum, mineral oils, (mixtures of oil-derived hydrocarbon oils), polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane, and their mixtures.

The total amount of oils can vary, but typically ranges from about 0.01% to about 10%, such as from about 0.1% to about 8%, or from about 0.5% to about 6% by weight, relative to the total weight of the base lightening composition. For example, in some embodiments, the total amount of oils may range from about 0.5% to about 5%, from about 0.5% to about 4%, from about 0.5% to about 3%, from about 0.5% to about 2.5%, from about 0.5% to about 2%, from about 0.5% to about 1.5%, from about 0.5% to about 1%, from about 0.75% to about 5%, from about 0.75% to about 4%, from about 0.75% to about 3%, from about 0.75% to about 2.5%, from about 0.75% to about 2%, from about 0.75% to about 1.5%, from about 0.75% to about 1%, from about 1% to about 5%, from about 1% to about 4%, from about 1% to about 3%, from about 1% to about 2.5%, from about 1% to about 2%, or from about 1% to about 1.5% by weight, relative to the total weight of the base lightening composition.

### Fillers

Optionally, base lightening compositions according to the disclosure comprise at least one filler (other than silicates). Non-limiting examples of fillers include starches, maltodextrins, perlites, zeolites, polylactic acids, silicas, polyamide powders, polyvinylpyrrolidones, dextrose, oligosaccharides, celluloses, diatomite, diatomaceous earth, talc, clays, silicon dioxide, clays, or mixtures thereof. In some instances, the one or more fillers comprises one or more alkali metal salts of fatty acids and/or organic base salts of fatty acids, for example, sodium stearate, zinc laurate, magnesium stearate, magnesium myristate, zinc stearate, ammonium stearate, ammonium oleate, ammonium nonanoate, sodium palmitate, potassium stearate, potassium palmitate, sodium myristate, aluminum monostearate, or combinations of two or more thereof.

If present, the total amount of fillers may vary, but typically ranges from about 1% to about 20%, including all subranges therebetween, such as from about 1% to about 18%, from about 2% to about 16%, from about 2.5% to about 15%, or from about 3% to about 14%, or from about 4% to about 13% by weight, relative to the total weight of the base lightening composition, for example from about 1% to about 18%, from about 1% to about 16%, from about 1% to about 15%, from about 1% to about 14%, from about 1% to about 13%, from about 1% to about 12%, from about 1.5% to about 20%, from about 1.5% to about 16%, from about 1.5% to about 15%, from about 1.5% to about 14%, from about 1.5% to about 13%, from about 1.5% to about 12%, from about 2% to about 20%, from about 2% to about 18%, from about 2% to about 15%, from about 2% to about 14%, from about 2% to about 13%, from about 2% to about 12%, from about 2.5% to about 20%, from about 2.5% to about 18%, from about 2.5% to about 16%, from about 2.5% to about 14%, from about 2.5% to about 13%, from about 2.5% to about 12%, from about 3% to about 20%, from about 3% to about 18%, from about 3% to about 16%, from about 3% to about 15%, from about 3% to about 13%, from about 3% to about 12%, from about 4% to about 20%, from about 4% to about 18%, from about 4% to about 16%, from about 4% to about 15%, from about 4% to about 14%, or from about 4% to about 12% by weight, relative to the total weight of the base lightening composition.

### Thickening Agents

The base lightening compositions can also optionally include one or more thickening agents. Non-limiting examples of thickening agents include carboxylic acid polymers, crosslinked polyacrylate polymers, polyacrylamide polymers, polysaccharides, and gums. More specific, non-limiting examples of thickening agents include acacia, agar, algin, alginic acid, ammonium alginate, amylopectin, calcium alginate, calcium carrageenan, carnitine, carrageenan, celluloses and modified celluloses such as hydroxyethylcellulose, dextrin, gelatin, gellan gum, guar gum, guar hydroxypropyltrimonium chloride, hydroxylpropyl guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, or mixtures thereof. In some cases, the one or more thickening agents include a crosslinked polyacrylate polymer, for example, acrylates/C10-30 alkylacrylate crosspolymer. Furthermore, in some cases the one or more thickening agents include guar gum.

If present, the total amount of thickening agents may range from about 0.01% to about 10%, such as from about 0.01% to about 5%, from about 0.1% to about 5%, from about 0.5% to about 5%, from about 0.5% to about 3%, from about 1% to about 5%, from about 1% to about 3%, from about 1.25% to about 2.75%, or from about 1.5% to about 2.5% by weight, relative to the total weight of the base lightening composition.

### Anionic Surfactants

Optionally, base lightening compositions comprise one or more anionic surfactants. For instance, the anionic surfactant(s) that may be useful include alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside sulfosuccinates.

Use is also made of (C₆-C₂₄)alkyl sulfates, (C₆-C₂₄)alkyl ether sulfates, which are optionally ethoxylated, comprising from 2 to 50 ethylene oxide units, or mixtures thereof, in particular in the form of alkali metal salts or alkaline-earth metal salts, ammonium salts or amino alcohol salts.

In some cases, the one or more anionic surfactant is selected from the group consisting of sodium oleyl succinate, ammonium lauryl sulphosuccinate, sodium lauryl sulfate, sodium lauryl ether sulfate (also known as sodium laureth sulfate, SLES), sodium lauryl ether sulphosuccinate, ammonium lauryl sulfate (ALS), ammonium lauryl ether sulfate (ammonium laureth sulfate), sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate, or mixtures thereof. In some instances, preferred anionic surfactants include sodium lauryl sulfate, sodium lauryl ether sulfate, sodium lauryl ether sulphosuccinate, ammonium lauryl sulfate, ammonium lauryl ether sulfate, or mixtures thereof.

If present, the total amount of anionic surfactants may range from about 0.1% up to about 15%, such as up to about 12%, up to about 10%, up to about 9%, up to about 8%, up to about 7%, or up to about 6% by weight, relative to the total weight of the base lightening composition. For example, the total amount of anionic surfactants may range from about 0.1% to about 10%, from about 0.5% to about 9%, from about 1% to about 8%, or from about 2% to about 7% by weight, relative to the total weight of the base lightening composition. In at least some embodiments, the base lightening compositions comprise at least one anionic surfactant, and have a total amount of anionic surfactants ranging from about 0.5% to about 12%, such as from about 1% to about 10%, from about 1.5% to about 9%, or from about 2% to about 8%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the base lightening composition, including any range using any of the foregoing as upper and lower limits.

The base lightening composition may comprise additional auxiliary components, for example preservatives, hair conditioning agents, vitamins, chelating agents, pigments or colorants to color the composition, or any other component typically used in hair color lightening base compositions.

In some embodiments the base lightening composition will be anhydrous or substantially anhydrous, for example with less than about 1% water, such as less than about 0.5% water, or less than about 0.1% water. The form of the base lightening composition may be a powder, cream, gel, or the like.

### Additive Compositions

In various embodiments, additive compositions according to the disclosure comprise (a) at least one carboxylic acid or salt thereof, (b) at least one cyclodextrin or derivative thereof, (c) at least one polysaccharide thickening agent, (d) at least one polyol, and (e) water.

### Carboxylic Acids or Salts Thereof

Additive compositions according to the disclosure comprise at least one carboxylic acid or salt thereof. Mixtures of carboxylic acids and/or salts of carboxylic acids may be chosen. As used herein, the term “carboxylic acid” includes salts of carboxylic acids whether or not expressly stated. The carboxylic acids which may be used include, in various embodiments, mono-carboxylic acids (one carboxylic group) or poly-carboxylic acids (two or more carboxylic groups). Preferably, the carboxylic acids are non-polymeric carboxylic acids. The carboxylic acids may be chosen, in certain embodiments, from those having a molecular weight of less than about 900 g/mol, such as less than about 700 g/mol, less than about 500 g/mol, less than about 300 g/mol, or less than about 200 g/mol.

In some embodiments, the at least one carboxylic acid may be chosen from mono-carboxylic acids such as formic acid, acetic acid, propionic acid, butyric acid, valeric acid, caproic acid, entanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, lactic acid, or salts thereof.

In further embodiments, the at least one carboxylic acid may be chosen from di-carboxylic acids such as oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, or salts thereof.

In yet further embodiments, the at least one carboxylic acid may be chosen from tri-carboxylic acids such as citric acid, isocitric acid, aconitric acid, propane-1,2,3-tricarboxylic acid, benzene-1,3,5-tricarboxylic acid, or salts thereof.

In still further embodiments, the at least one carboxylic acid may be chosen from poly-carboxylic acids that have more than three carboxylic groups, such as phytic acid.

In various embodiments, the additive compositions comprise a mixture of two or more carboxylic acids and/or salts thereof. For example, the additive compositions may comprise a mixture of two or more mono-carboxylic acids and/or salts thereof, or may comprise a mixture or two or more poly-carboxylic acids and/or salts thereof, such as two or more di-carboxylic acids, two or more tri-carboxylic acids, or a mixture of one or more di-carboxylic acids and one or more tri-carboxylic acids, and so on. Mixtures of one or more mono-carboxylic or salts thereof with one or more poly-carboxylic acids or salts thereof are also contemplated. In further embodiments, the additive composition comprises only one carboxylic acid or a salt thereof.

In certain embodiments, the at least one carboxylic acid is chosen from citric acid, lactic acid, malic acid, maleic acid, tartaric acid, glycolic acid, phytic acid, salts thereof, or a mixture of two or more of the foregoing.

In various embodiments, the additive compositions comprise a total amount of carboxylic acids and/or salts thereof ranging from about 0.01% to about 20% by weight, such as about 0.1% to about 18%, about 0.5% to about 15%, or about 1% to about 13% by weight, relative to the total weight of the additive composition. By way of non-limiting example, the additive compositions may comprise a total amount of carboxylic acids and/or salt thereof ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5%, by weight, relative to the total weight of the additive composition, including all ranges and subranges thereof. In some embodiments, the additive compositions comprise a total amount of carboxylic acids and/or salts thereof ranging from about 0.5% to about 13%, such as from about 0.5% to about 10%, or from about 0.5% to about 8% by weight, relative to the total weight of the additive composition.

In some embodiments, the at least one carboxylic acid comprises or consists of citric acid, lactic acid, malic acid, maleic acid, tartaric acid, glycolic acid, phytic acid, salts thereof, or a mixture thereof, in an amount ranging from about 0.1% to about 10%, such as from about 0.3% to about 9%, or from about 0.5% to about 8% by weight, relative to the total weight of the additive composition, including all ranges and subranges thereof.

In still further embodiments, the additive composition comprises citric acid and/or salts thereof in an amount ranging from about 1% to about 5%, such as from about 1% to about 4%, from about 1% to about 3%, from about 1.5% to about 4%, from about 1.5% to about 3.5%, from about 1.5% to about 3%, from about 2% to about 4%, from about 2% to about 3.5%, from about 2% to about 3%, or from about 2.25% to about 2.75%, including all ranges and subranges thereof, or is about 1%, about 1.5%, about 2%, about 2.5%, about 3%, or about 3.5% by weight, relative to the total weight of the additive composition, including all ranges and subranges using any of the foregoing as upper or lower limits.

### Cyclodextrins or Derivatives Thereof

Additive compositions according to the disclosure comprise at least one cyclodextrin or derivative thereof. As used herein, the term “cyclodextrins” includes derivatives, whether or not expressly stated. Cyclodextrins are a family of cyclic oligosaccharides consisting of a macrocyclic ring of glucose subunits joined by α-1,4 glycosidic bonds.

The cyclodextrins that can be used include those of formula (III):

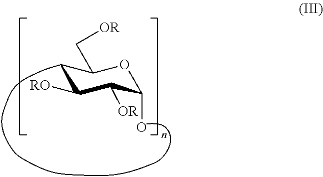

wherein R is chosen from H, CH₃, or a hydroxypropyl group, and n ranges from 6-8.

For example, in embodiments where R=H, the cyclodextrin may be α-cyclodextrin (n=6), ß-cyclodextrin (n=7), or γ-cyclodextrin (n=8). By way of example, α-cyclodextrin sold by the company WACKER under the name CAVAMAX W6 PHARMA, ß-cyclodextrin sold by the company WACKER under the name CAVAMAX W7 PHARMA, or γ-cyclodextrin sold by the company WACKER under the name CAVAMAX W8 PHARMA can be used.

In other embodiments where R=CH₃, the cyclodextrin may be a methyl-cyclodextrin, such as methyl-α-cyclodextrin (n=6), methyl-ß-cyclodextrin (n=7), or methyl-γ-cyclodextrin (n=8). For example, the methyl-ß-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodextrin may be a mixture of α-cyclodextrin, ß-cyclodextrin, and/or γ-cyclodextrin. In another embodiment, the at least one cyclodextrin includes ß-cyclodextrin. In yet a further embodiment, the cyclodextrin is only ß-cyclodextrin, and no other cyclodextrins or derivatives thereof are present in the additive composition.

In various embodiments, the additive compositions comprise a total amount of cyclodextrins and/or derivatives thereof in an amount ranging from about 0.01% to about 20% by weight, including all ranges and subranges thereof, such as from about 0.1% to about 18%, from about 0.5% to about 18%, from about 0.5% to about 15%, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 18%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the additive composition. In some embodiments, the additive compositions comprise a total amount of cyclodextrins and/or derivatives thereof ranging from about 1% to about 20%, such as from about 0.1% to about 18%, from about 0.5% to about 15%, from about 0.5% to about 10%, from about 0.5% to about 8%, from about 1% to about 13%, from about 1% to about 7%, or from about 1% to about 5% by weight, relative to the total weight of the additive composition.

In some embodiments, the additive compositions comprises ß-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the additive composition, including all ranges and subranges thereof.

In various embodiments, it is particularly advantageous to choose total amounts of carboxylic acids (including salts) and cyclodextrins (including derivatives) such that the mole ratio of carboxylic acid(s):cyclodextrin(s) in the additive composition ranges from about 0.5:1 to about 25:3, for example about 4:1 to about 15:1, about 5:1 to about 12:1, or about 6:1 to about 10:1, such as, for example, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits.

In some embodiments, additive compositions according to the disclosure may have a weight ratio of (a) the total amount of carboxylic acids and salts thereof to (b) the total amount of cyclodextrins and derivatives thereof ranging from about 1:3 to about 6:1, for example from about 1:3 to about 3:1, from about 1:2.5 to about 2.5:1, from about 1:2 to about 2:1, from about 1:1 to about 1.75:1, from about 1.5:3 to about 3:1.5, from about 1.5:2.5 to about 2.5:1.5, or from about 1.5:2 to about 2:1.5, or may be about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, about 6:1, about 1.5:3, about 1.5:2.5, about 1.5:2, about 2:1.1, about 2.5:1.5, about 3:1.5, about 3.5:1.5, about 4:1.5, about 4.5:1.5, about 5:1.5, about 5.5:1.5, or about 6:1.5, including all ranges and subranges using any of the foregoing as upper or lower limits.

### Polysaccharide Thickening Agents

Compositions according to the disclosure comprise at least one polysaccharide as a thickening agent. The term “thickening agent” may be referred to interchangeably to as “thickener” or “viscosity modifying agent.” The at least one polysaccharide agent may be chosen from anionic polysaccharide thickening agents, nonionic polysaccharide thickening agents, or mixtures of two or more thereof. Polysaccharide thickening agents that can be used include, by way of example only, inulins, celluloses, starches, gums, or derivatives of the foregoing.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, dehydroxanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxymethylcellulose, methylhydroxyethylcellulose, hydroxyethylcelluloses, hydroxypropylmethylcellulose, hydroxybutylcellulose, hydroxyethylmethylcellulose (also known as methyl hydroxyethylcellulose) or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, preferably nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose. Other particular thickening agents suitable for use in the additive compositions described herein are hydroxypropyl guar gums.

Non-limiting examples of starches include modified starches, starch-based polymers, methylhydroxypropyl starch, potato starch, modified potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In some embodiments, the polysaccharide thickening agents are chosen from carrageenan, xanthan gum, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropylguar gum, hydroxyethyl guar gum, hydroxypropyl guar, xylose gum, cellobiose, dextrin, maltodextrin, scleroglucan, chitosan, ulvan, fucoidan, alginate, pectin, heparin, inulin, levan, or mixtures thereof. In further embodiments, the polysaccharide thickening agents are chosen from sclerotium gum, carrageenan, xanthan gum, cellulose gum, cyclodextrin, or mixtures of two or more thereof.

In various exemplary embodiments, the total amount of polysaccharide thickening agents may vary, and may typically range from about 0.05% to about 5%, such as from about 0.1% to about 5%, from about 0.1% to about 4%, from about 0.1% to about 3%, from about 0.1% to about 2%, from about 0.1% to about 1%, from about 0.5% to about 5%, from about 0.5% to about 4%, from about 0.5% to about 3%, from about 0.5% to about 2%, from about 0.5% to about 1%, from about 1% to about 5%, from about 1% to about 4%, from about 1% to about 3%, or from about 1% to about 2% by weight, relative to the total weight of the additive composition, including all ranges and subranges thereof. In some embodiments, the total amount of polysaccharide thickening agents may range from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the additive composition, including all ranges and subranges using any of the foregoing as upper or lower limits.

For example, the additive compositions may, in exemplary embodiments, comprise a total amount of polysaccharide thickening agents ranging from about 0.1% to about 5%, from about 0.5% to about 4%, or from about 0.75% to about 3%, preferably from about 0.5% to about 2% by weight, relative to the total weight of the additive composition, wherein the polysaccharide thickening agent comprises at least one polysaccharide thickening agent may be chosen from gum arabic, tragacanth gum, karaya gum, guar gum, gellan gum, tara gum, locust bean gum, tamarind gum, xanthan gum, dehydroxanthan gum, Seneca gum, sclerotium gum, and derivatives thereof. In various embodiments, the additive composition comprises guar gum and/or a derivative thereof in an amount ranging from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the additive composition, including all ranges and subranges using any of the foregoing as upper or lower limits.

### Polyols

Compositions according to the disclosure comprise at least one polyol. By way of example only, the at least one polyol may be chosen from C₂-C₁₆ polyols, such as C₂-C₁₂ or C₂-C₈ polyols. The at least one polyol may optionally be chosen from diols and triols. The at least one polyol may be linear or branched, saturated or unsaturated, and substituted or unsubstituted. Any stereoisomer of the polyols may also be used.

In various exemplary embodiments, the polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, ethylhexyglycerin, or mixtures of two or more thereof.

In other embodiments, the polyols may be chosen from, for example, propanediol, butanediol, pentanediol, hexanediol, heptanediol, octanediol, 1,2,6-hexanetriol, 1,2,4-butanetriol, trimethylolpropane, 2-butene-1,4-diol, 2-ethyl-1,3-hexanediol, 2-methyl-2,4-pentanediol, caprylyl glycol, 1,2-hexanediol, 1,2-pentanediol, 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, 2-ethyl-1,3-hexanediol, 4-methyl-1,2-pentanediol, or mixtures of two or more thereof.

In some embodiments, the additive compositions comprise at least two polyols, for example at least two polyols chosen from diols and/or triols. As one non-limiting example, an additive composition according to the disclosure may comprise a combination of at least one glycerin compound and at least one additional glycol compound.

In various embodiments, the total amount of polyols can be about 1% or more, such as about 2% or more, about 3% or more, about 4% or more, about 5% or more, about 6% or more, for example up to about 10% by weight, based on the total weight of the additive composition. In some embodiments, the total amount of polyol is present in the additive composition with a total amount ranging from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 8%, from about 0.5% to about 7%, from about 0.5% to about 6%, from about 0.5% to about 5%, from about 0.5% to about 4% by weight, from about 0.5% to about 3%, from about 0.5% to about 2%, or from about 0.5% to about 1.5% by weight, relative to the total weight of the additive composition. In some embodiments, the total amount of polyols ranges from about 0.1% to about 5%, such as from about 0.25% to about 4%, from about 0.5% to about 3%, or from about 0.5% to about 2.5%, or may be about 1%, about 1.5%, about 2%, about 2.5%, about 3%, or about 3.5% by weight, relative to the total weight of the additive composition, including all ranges and subranges using any of the foregoing as upper or lower limits.

In some exemplary and non-limiting embodiments, the additive compositions comprise at least one polyol chosen from diols and/or triols, and the total amount of polyols is about 1% or more, about 1.5% or more, about 2% or more, about 3% or more, about 4% or more, or about 5% or more by weight, for example up to about 10% by weight, relative to the total weight of the additive composition. In various embodiments, the additive comprises glycerin.

### Water

Additive compositions according to the disclosure comprise water. In various embodiments, the amount of water in the additive composition is generally greater than 50%, and may, for example, range from about 70% to about 98%, including all subranges therebetween, such as from about 75% to about 98%, from about 80% to about 98%, from about 85% to about 98%, from about 90% to about 98%, from about 95% to about 98%, from about 70% to about 95%, from about 75% to about 95%, from about 80% to 95%, from about 85% to 95% by weight, relative to the total weight of the additive composition.

### Additional Components

Compositions according to the disclosure may optionally comprise one or more additional components suitable for use in such compositions. For example, the additive compositions may comprise pH adjusters, preservatives, solvents other than water, and/or one or more auxiliary components. Optional additional components may be present individually or collectively in the additive composition in amounts ranging up to about 15%, such as from about 0.001% to about 10%, from about 0.01% to about 5%, or from about 0.01% to about 1% by weight, relative to the total weight of the additive composition.

In various embodiments, the additive compositions are acidic and have a pH of less than 7. For example, the pH of the additive composition may range from about 1.5 to about 6.8, from about 2 to about 6.5, from about 2.5 to about 6, from about 3 to about 6.5, from about 3 to about 6, from about 3 to about 5.5, from about 3 to about 5, from about 3 to about 4, from about 3.5 to about 6.5, from about 3.5 to about 6, from about 3.5 to about 5.5, from about 3.5 to about 5, from about 3.5 to about 4.5, or from about 3.5 to about 4, including all ranges and subranges using any of the foregoing as upper or lower limits. In some exemplary embodiments, the pH of the additive composition ranges from about 3 to about 6.5. In further exemplary embodiments, the pH of the additive composition ranges from about 3 to about 4.

Therefore, the additive compositions may include one or more pH adjusters to increase or decrease the overall pH of the additive composition. For example, one or more acids may be included to decrease the pH of the additive composition. Examples of suitable acids for decreasing the pH of the additive compositions may include one or more acids. The additive compositions may include one or more bases, such as sodium hydroxide, potassium hydroxide and the like, to increase the pH of the additive composition.

One or more preservatives may be included in the additive compositions. Suitable preservatives include, but are not limited to, glycerin containing compounds (e.g., glycerin or ethylhexylglycerin or phenoxyethanol), benzyl alcohol, parabens(methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), or mixtures thereof. In some cases, the additive compositions may include one or more preservatives selected from the group consisting of sodium benzoate, benzoic acid, chlorhexidine digluconate, chlorhexidine dihydrochloride, salicylic acid, phenoxyethanol, methyl paraben, or mixtures thereof.

The total amount of the one or more preservatives in the additive compositions, when present, may vary. In some cases, the total amount of the one or more preservatives may range from about 0.01% to about 5%, from about 0.01% to about 4%, from about 0.01% to about 2%, from about or about 0.01% to about 3% by weight, relative to the total weight of the additive composition.

The additive compositions may optionally comprise any auxiliary component suitable for use in such compositions. Such components may include, but are not limited to, dyes/pigments for adding color to the composition, moisturizing agents, fatty substances, thickeners other than those previously described, fillers, structuring agents, shine agents, antioxidants or penetrants, sequestrants, fragrances, buffers, dispersants, plant extracts, such as apricot seed powder, opacifiers, sunscreen agents, vitamins, and antistatic agents.

### Pre-Phase Mixture

Although not required, in some embodiments it may be advantageous to prepare the additive composition by combining and mixing the carboxylic acid(s) and cyclodextrin(s) with specific mole and/or ratios to allow the carboxylic acid(s) and cyclodextrin(s) to form aggregates. The pre-phase mixture can then be mixed with additional components such as polysaccharide(s), polyol(s), optional additives, and water, to form the additive composition.

In various embodiments, in the pre-phase mixture, it may be particularly advantageous to choose total amounts of carboxylic acids (including salts) and cyclodextrins (including derivatives) so that a mole ratio of carboxylic acid(s):cyclodextrin(s) ranges from about 0.5:1 to about 25:3, preferably from about 4:1 to about 15:1, from about 5:1 to about 12:1, or from about 6:1 to about 10:1, or may be present in mole ratios of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 3:2, about 5:2, about 7:2, about 9:2, about 11:2, about 13:2, about 15:2, about 17:2, about 19:2, about 1:3, about 2:3, about 4:3, about 5:1, about 7:3, about 8:3, about 10:3, about 11:3, about 13:3, about 14:3, about 16:3, about 17:3, about 19:3, or about 20:3, including all ranges and subranges using any of the foregoing as upper or lower limits.

Correspondingly, in the pre-phase mixture, it may be particularly advantageous to choose total amounts of carboxylic acids (including salts) and cyclodextrins (including derivatives) so that a weight ratio of the total amount of carboxylic acids and salts thereof to the total amount of cyclodextrins and derivatives thereof ranges from about 1:3 to about 6:1, or may be present in weight ratios of about 1:3, about 1:2.5, about 1:2, about 1:1.5, about 1:1, about 1.5:1, about 2:1, about 2.5:1, about 3:1, about 3.5:1, about 4:1, about 4.5:1, about 5:1, about 5.5:1, or about 6:1, including all ranges and subranges using any of the foregoing as upper or lower limits.

Once the particular carboxylic acid(s), cyclodextrin(s), and solvent(s), as well as the desired mole and/or weight ratios thereof, are chosen, the pre-mixture may be prepared by any conventional method.

In various embodiments, additive compositions according to the disclosure may contain a weight ratio of pre-mixture:additional components ranging from about 1:99 to about 50:50. For example, additive compositions according to the disclosure may contain a weight ratio of pre-mixture:additional components ranging from about 2:98 to about 40:60 or about 3:97 to about 35:65, for example may be about 2.5:97.5, about 5:95, about 7.5:92.5, about 10:90, about 12.5:87.5, about 15:85, about 17.5:81.5, about 20:80, about 25:75, or about 30:70, including all ranges and subranges using any of the foregoing as upper or lower limits. In preferred embodiments, the weight ratio of pre-mixture:additional components in the additive composition ranges from about 5:95 to about 15:85.

### Oxidizing Compositions and Oxidizing Agents

The oxidizing composition (developer) or oxidizing agent that can be mixed with the base lightening composition or mixture to prepare the hair lightening composition at or near the time of use is not limited, and any conventional oxidizing composition or oxidizing agent for lightening hair can be used. Typically an oxidizing composition comprises an oxidizing agent such as peroxide, inorganic perhydrate salts, or a source of peroxymonocarbonate ions, in a cosmetically acceptable carrier. Non-limiting examples of peroxides include hydrogen peroxide, inorganic alkali metal peroxides such as sodium peroxide, and organic peroxides such as urea peroxide and melamine peroxide. Non-limiting examples of inorganic perhydrate salts include the alkali metal salts of perborates, percarbonates, perphosphates, persilicates, persulphates, and the like. These inorganic perhydrate salts may optionally be used as monohydrates, tetrahydrates, etc.

As non-limiting examples of sources of peroxymonocarbonate ions, in situ formation from a source of hydrogen peroxide and a source of hydrogen carbonate ions can be used. This system is particularly effective in combination with a source of ammonia or ammonium ions. Accordingly, any source of peroxymonocarbonate ions may be utilized. Suitable sources include sodium, potassium, guanidine, arginine, lithium, calcium, magnesium, barium, ammonium salts of carbonate, carbamate and hydrocarbonate ions and mixtures thereof such as sodium carbonate, sodium hydrogen carbonate, potassium carbonate, potassium hydrogen carbonate, guanidine carbonate, guanidine hydrogen carbonate, lithium carbonate, calcium carbonate, barium carbonate, ammonium carbonate, ammonium hydrogen carbonate, and mixtures thereof. Percarbonate salts may also be utilized to provide both the source of carbonate ions and as an oxidizing agent. Non-limiting examples of carbonate ions, carbamate, and hydrocarbonate ions are sodium hydrogen carbonate, potassium hydrogen carbonate, ammonium carbamate, and mixtures of two or more thereof.

The amount of oxidizing agent will vary, but typically ranges from about 1% to about 40%, such as from about 2% to about 30%, from about 2% to about 25%, from about 2% to about 20%, from about 2% to about 15%, from about 2% to about 10%, from about 2% to about 5%, from about 5% to about 30%, from about 5% to about 25%, from about 5% to about 20%, from about 5% to about 15%, from about 5% to about 10%, from about 10% to about 30%, from about 10% to about 25%, from about 10% to about 20%, or from about 10% to about 15% by weight, relative to the total weight of the oxidizing composition, including any range using any of the foregoing as upper and lower limits. By way of example only, the oxidizing agent may comprise, consist essentially of, or consist of hydrogen peroxide, present in any of the foregoing ranges, for example about 3%, about 6%, about 12%, or about 18% by weight, relative to the total weight of the oxidizing composition. However, because oxidizing agents also damage the hair, it would be advantageous to reduce the amount needed to achieve the same or even greater levels of lightening.

The oxidizing composition may further comprise additional components such as thermal control agents as described above, oils as described above, anionic surfactants as described above, thickening agents as described above, organic acids and their salts as described above, amino acids and their salts as described above, nonionic surfactants, cationic surfactants, amphoteric surfactants, hair conditioning agents, preservatives, vitamins, chelating agents, pigments or colorants to color the composition, pH adjusters, fragrances, or any other component typically used in developer compositions for hair lightening. In a preferred embodiment, the oxidizing composition is free or essentially free of alkanolamines.

The oxidizing composition may be in the form of a powder, gel, liquid, foam, lotion, cream, mousse, or emulsion. In some embodiments, the oxidizing composition is anhydrous, and is mixed with water prior to or simultaneously with being mixed with a base lightening composition or mixture. The oxidizing composition may optionally be aqueous, and have a pH less than 7, such as ranging from about 1 to about 6 or about 2 about 5. For example, the oxidizing composition may be a 10V, 20V, 30V, or 40V aqueous hydrogen peroxide composition, and have a pH ranging from about 2.5 to about 4.5.

### Base Lightening Mixtures

As discussed, in some embodiments the base lightening compositions according to the disclosure can be mixed with an appropriate amount of the additive composition to form a base lightening mixture, which can then be mixed with an oxidizing composition or oxidizing agent in a desired mix ratio to form a hair lightening composition. In some embodiments, an additional amount of water can also be added to or mixed with the base lightening mixture.

The amount of additive composition to be mixed with or added to the base lightening composition to prepare the base lightening mixture can be determined by the amount of persulfates and alkalizers present. Typically, traditional base lightening compositions will comprise in the range of 50-60% persulfates. Thus, for example, a base lightening composition comprising 35% persulfates and an additional 5% of alkalizers could, in an exemplary embodiment, be mixed with an amount of additive composition in the range of 10-20% by weight, relative to the total weight of the base lightening composition, as a superaddition.

In various embodiments, the amount of additive composition that may be mixed with or added to the base lightening composition can range from about 1% to about 25%, such as, for example, from about 1% to about 20%, from about 1.5% to about 18%, from about 2% to about 15%, from about 2.5% to about 12%, from about 3% to about 10%, from about 3.5% to about 8%, from about 4% to about 6%, or may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 20.5%, about 21%, about 21.5%, about 22%, about 22.5%, about 23%, about 23.5%, about 24%, about 24.5%, or about 25% by weight, relative to the total weight of the base lightening composition, including all ranges and subranges using any of the foregoing as upper and lower limits.

In other embodiments, the amount of additive composition mixed with the base lightening composition may be chosen relative to the total amount of persulfates in the base lightening composition. For example, the weight ratio of total persulfates in the base lightening composition to the amount of additive composition may range from about 1:1 to about 10:1, such as from about 1:1 to about 5:1, or from about 1.5:1 to about 3.5:1. In various embodiments, the weight ratio of the total amount of persulfates in the base lightening composition to the amount of additive composition mixed with the base lightening composition may be about 1:1, about 1.25:1, about 1.5:1, about 1.75:1, about 2:1, about 2.25:1, about 2.5:1, about 2.75:1, about 3:1, about 3.25:1, about 3.5:1, about 3.75:1, about 4:1, about 4.25:1, about 4.5:1, about 4.75:1, or about 5:1, or may be any range using any of the foregoing as upper and lower limits.

In other embodiments still, the amount of additive composition added to or mixed with the base lightening composition may be chosen relative to the total weight of the base lightening composition. For example, the weight ratio of total weight of the base lightening composition to additive composition may range from about 5:1 to about 15:1, such as from about 6:1 to about 14:1, or from about 7:1 to about 13.5:1. In various embodiments, the weight ratio of the total weight of the base lightening composition to the amount of additive composition mixed with the base lightening composition may be about 5:1, about 5.25:1, about 5.5:1, about 5.75:1, about 6:1, about 6.25:1, about 6.5:1, about 6.75:1, about 7:1, about 7.25:1, about 7.5:1, about 7.75:1, about 8:1, about 8.25:1, about 8.5:1, about 8.75:1, about 9:1, about 9.25:1, about 9.5:1, about 9.75:1, about 10:1, about 10.25:1, about 10.5:1, about 10.75:1, about 11:1, about 11.25:1, about 11.5:1, about 11.75:1, about 12:1, about 12.25:1, about 12.5:1, about 12.75:1, about 13:1, about 13.25:1, about 13.5:1, about 13.75:1, about 14:1, about 14.25:1, about 14.5:1, about 14.75:1, or about 15:1, or may be any range using any of the foregoing as upper and lower limits.

In still further embodiments, the amount of additive composition added to or mixed with the base lightening composition may be chosen to provide a specific weight ratio of cyclodextrins to persulfates in the base lightening mixture. For example, the weight ratio of cyclodextrins to persulfates in the base lightening mixture may range from about 0.001:1 to about 0.5:1, such as from about 0.001:1 to about 0.4:1, about 0.001:1 to about 0.3:1, about 0.001:1 to about 0.2:1, about 0.001:1 to about 0.1:1, about 0.002:1 to about 0.5:1, about 0.002:1 to about 0.4:1, about 0.002:1 to about 0.3:1, about 0.002:1 to about 0.2:1, about 0.002:1 to about 0.1:1, about 0.003:1 to about 0.5:1, about 0.003:1 to about 0.4:1, about 0.003:1 to about 0.3:1, about 0.003:1 to about 0.2:1, about 0.003:1 to about 0.1:1, about 0.004:1 to about 0.5:1, about 0.004:1 to about 0.4:1, about 0.004:1 to about 0.3:1, about 0.004:1 to about 0.2:1, about 0.004:1 to about 0.1:1, about 0.005:1 to about 0.5:1, about 0.005:1 to about 0.4:1, about 0.005:1 to about 0.3:1, about 0.005:1 to about 0.2:1, or about 0.005:1 to about 0.1:1.

Although not necessary, it may be advantageous for the user to combine the base lightening composition and additive composition to prepare the base lightening mixture at or near the time of use, for example just before or simultaneously with mixing the base lightening composition and oxidizing composition/oxidizing agent. Alternatively, the user could combine the additive composition and the oxidizing composition, and subsequently combine that mixture with the base lightening composition. It should be understood that the order of mixing is not critical. Alternatively, the additive composition may be combined with the base lightening composition and/or oxidizing composition during manufacture.

### Hair Lightening Compositions

Hair lightening compositions according to the disclosure comprise a three-part system of (1) a base lightening compositions according to the disclosure, (2) an additive composition, and (3) an oxidizing composition or oxidizing agent. In various embodiments, the hair lightening compositions are prepared by mixing (1) a base lightening compositions according to the disclosure, (2) an additive composition, and (3) an oxidizing composition or oxidizing agent, for example at or near the time of use.

In traditional hair lightening compositions, a base lightening composition and an oxidizing composition (developer) are mixed at or near the time of use in a predetermined mix ratio, depending on strength and level of lift to be achieved. Such predetermined ratios typically range from about 1:3 to about 3:1, such as from about 1:2.5 to about 2.5:1, from about 1:2 to about 2:1, or from about 1:1.5 to about 1.5:1, for example about 1:1. In embodiments where the additive composition is mixed with the base lightening composition to form a base lightening mixture, the base lightening mixture and oxidizing composition may be mixed at or near the time of use, for example in a mix ratio of [base lightening mixture]:[oxidizing composition] ranging from about 1:3 to about 3:1, such as from about 1:2.5 to about 2.5:1, from about 1:2 to about 2:1, or from about 1:1.5 to about 1.5:1, such as about 1:1, to form the hair lightening composition. For example, the mix ratio of [base lightening mixture]:[oxidizing composition] may be about 1:1, about 1:1.5, or about 1:2.

As such, the amounts of components described herein for the base lightening composition, additive composition, and oxidizing composition will be present in the hair lightening composition in amounts consistent with the chosen mix ratio. By way of illustration only, if a 1:1 mix ratio of [base lightening mixture]:[oxidizing composition] is used, the amounts of components in the oxidizing composition will be present in the hair lightening composition at 50% of the amounts described herein.

The hair lightening composition will typically have a pH ranging from about 8 to about 13, such as from about 9 to about 12, from about 9.5 to about 11, from about 10 to about 11, or from about 10 to about 10.5.

In one embodiment, the hair lightening composition is free or essentially free of alkanolamines, although derivatives of alkanolamines may be included in such hair lightening compositions. In preferred embodiments, the hair lightening composition is free or essentially free of monoethanolamine.

Surprisingly, hair lightening compositions according to the disclosure are surprisingly effective at providing high or very high levels of lightening in a shorter period of time compared to traditional hair lightening compositions, despite the reduced amounts of persulfates. In addition, the treated hair has less damage, is stronger, and appears and feels healthier compared to hair treated with traditional hair lightening compositions. The treated hair also has less frizz, less volume, and, in the case of curly or wavy hair, better retention of the curl or wave shape.

The hair lightening compositions are stable and have a smooth, light, whip cream-like texture upon mixing the base lightening composition or base lightening mixture and developer, which texture remains during the lightening process. This is in contrast to traditional hair lightening mixtures which become thick during application and solid and crumbly during the lightening process.

Hair lightening compositions according to the disclosure effectively control the peak temperature that is attained during the lightening process. In various embodiments, the peak temperature is less than 70° C., for example less than about 68° C., less than about 65° C., less than about 63° C., less than about 60° C., less than about 58° C., less than about 55° C., less than about 53° C., less than about 50° C., less than about 48° C., less than about 45° C., less than about 43° C., or less than about 40° C.

### II. Methods

The disclosure further relates to methods of preparing hair lightening compositions and methods of lightening hair. The methods comprising mixing a base lightening composition according to the disclosure, an additive composition according to the disclosure, and an oxidizing composition to form the hair lightening composition, and applying the hair lightening composition to the hair.

Methods of preparing hair lightening compositions according to the disclosure may, in various embodiments, comprise one or two mixing steps performed by the colorist or user. For example, a two-step method may comprise first mixing a base lightening composition with an additive composition to form a base lightening mixture, and second mixing the base lightening mixture with an oxidizing composition to form the hair lightening composition. An alternative method of preparing a hair lightening composition may comprise first mixing the additive composition with the oxidizing composition, and subsequently adding or mixing the base lightening composition therewith. As yet another example, methods of preparing a hair lightening composition may comprise first mixing a base lightening composition with an oxidizing composition, and subsequently adding or mixing the additive composition therewith.

In an exemplary embodiment, a method of preparing a hair lightening composition according to the disclosure comprises mixing (1) a base lightening composition comprising (a) from about 5% to about 20%, for example from about 8% to about 15%, of ammonium persulfate, (b) from about 10% to about 40%, for example from about 15% to about 35%, of one or more additional persulfate compounds, and (c) from about 5% to about 50% of one or more silicate compounds, (2) an amount of additive composition ranging from about 1% to about 25%, for example from about 2% to about 15%, and (3) an oxidizing agent, for example an aqueous 10V, 15V, 20V, 25V, 30V, 35V, or 40V hydrogen peroxide developer, where the weight ratio of the total amount of ammonium persulfate to the total amount of additional persulfate compounds in the base lightening composition is equal to or less than about 1, for example ranges from about 0.1 to about 0.9, and where all amounts are by weight relative to the total weight of the base lightening composition. The components (1), (2), and (3) may be mixed in any order, and in a mix ratio of [(1)+ (2)]:(3) ranging from about 1:3 to about 3:1, such as about 1:2 to about 2:1, about 1:1.5 to about 1.5:1, or about 1:1.

In another exemplary embodiment, a method of preparing a hair lightening composition according to the disclosure comprises mixing (1) a base lightening composition comprising (a) from about 10% to no more than 45% by weight, relative to the total weight of the base lightening composition, for example from about 15% to no more than 43%, of one or more persulfate compounds, and (b) from about 5% to about 50% of one or more silicate compounds, (2) an additive composition, and (3) an oxidizing agent, for example an aqueous 10V, 15V, 20V, 25V, 30V, 35V, or 40V hydrogen peroxide developer, where the weight ratio of the total amount of (a) persulfate compounds in the (1) base lightening composition to the total amount of (2) additive composition ranges from about 1:1 to about 1:10, for example from about 1:1 to about 5:1, or from about 1.5:1 to about 3.5:1. The components (1), (2), and (3) may be mixed in any order, and in a mix ratio of [(1)+ (2)]:(3) ranging from about 1:3 to about 3:1, such as about 1:2 to about 2:1, about 1:1.5 to about 1.5:1, or about 1:1.

In an exemplary one-step method, the additive composition and base lightening composition may be mixed during manufacture, and the pre-mixed base lightening mixture will be provided to the colorist or user. Thus, the colorist or user will mix the base lightening mixture and oxidizing composition at the desired mix ratio at or near the time of use to prepare the hair lightening composition.

Methods of lightening the color of hair are known. Typically, after the base lightening composition and developer composition are mixed, the hair lightening composition is applied to the hair (e.g., by brush, foam applicator, etc.), optionally the treated hair is covered, e.g., with foil, the composition allowed to remain on the hair for a time sufficient to achieve the desired alteration in the color of the hair (processing time), and then the composition is removed from the hair, e.g., by wiping, rinsing, and/or shampooing the hair.

Processing times needed to achieve the desired lightening of the color of the hair, e.g., desired degree or level of lift of the color of the hair vary. Exemplary processing times may range from about 5 minutes to about 75 minutes, such as about 10 minutes to about 70 minutes, about 15 minutes to about 65 minutes, about 20 minutes to about 60 minutes, about 25 minutes to about 55 minutes, about 30 minutes to about 50 minutes, or about 30 minutes to about 40 minutes, including all ranges and subranges using any of the aforementioned as upper and lower limits. In preferred embodiments, the processing time is less than 60 minutes, such as less than 55 minutes, for example is 50 minutes, 45 minutes, 40 minutes, 35 minutes, 30 minutes, or 25 minutes.

Surprisingly, the amount of time needed to achieve the same or even higher levels of lift with hair lightening compositions according to the disclosure is reduced compared to traditional hair lightening compositions. For example, the processing time to achieve the same or even higher levels of lift may be reduced by about or at least 5%, about or at least 10%, about or at least 15%, about or at least 20%, about or at least 25%, or about or at least 30%. In preferred embodiments, the processing time ranges from at least 20 minutes to less than 60 minutes.

Optionally, processing may take place at room temperature or at elevated temperatures (e.g., under a hood dryer).

Thus, in various embodiments, methods according to the disclosure comprise applying a hair lightening composition to the keratin materials to be treated, e.g. hair growing from the scalp, leaving the hair lightening composition on the hair for a processing time as described above, for example ranging from about 20 minutes to about 60 minutes, or about 30 minutes to about 50 minutes, with or without the use of heat and/or foils, and subsequently removing the hair lightening composition from the hair.

It is also possible to treat the hair with the hair lightening composition more than one time, e.g. two times (double processing) or more. In such embodiments, the composition will be applied to the hair, left on the hair for a first processing time and then removed from the hair (with or without rinsing and/or shampooing, for example wiped with a towel). Subsequently, a second hair lightening composition can be applied to the hair and left on the hair for a desired length of time. A further rinsing or wiping and reapplication can be done, or the hair can be rinsed and dried. The first and second lightening treatments can occur within the same service (e.g. within one to four hours of each other) or within separate but proximate services (e.g. within one to four days of each other), and can be the same or can be different. For example, the same hair lightening composition can be used in the first processing and second processing, and the processing times and conditions (e.g. heat or no heat) can be the same or different. As another example, two different hair lightening compositions can be used in the first processing and second processing, and the processing times and conditions (e.g. heat or no heat) can be the same or different.

In additional embodiments, the methods may comprise improving hair fiber integrity or reducing hair damage caused by lightening the hair by using hair lightening compositions according to the disclosure, compared to the damage caused by conventional hair lightening compositions. In further embodiments, the methods may comprise reducing loss of hair curl caused by lightening the hair by using hair lightening compositions according to the disclosure, compared to the damage caused by conventional hair lightening compositions.

### III. Kits

The disclosure further relates to kits that can be used in methods of lightening hair. The kits comprise at least two compartments or containers, such as at least three compartments or containers or more. The first compartment or container comprises a base lightening composition according to the disclosure. The second compartment or container comprises an additive composition according to the disclosure. Optionally, a third compartment or container may comprise an oxidizing composition. Alternatively, a third compartment or container may comprise a separate hair treatment composition, for example comprising a pre- and/or post-treatment hair conditioning composition.

In an exemplary embodiment, the kit comprises at least three compartments or containers, where the first compartment or container comprises a base lightening composition according to the disclosure, a second compartment or container comprises an additive composition according to the disclosure, and a third compartment or container comprises an oxidizing composition. The kit may optionally comprise an implement to mix the base composition, additive composition, and oxidizing composition, and/or to apply the mixture to the hair. In such an embodiment, the hair lightening composition prepared by mixing the contents of the first, second, and third compartments or containers would result in a hair lightening composition according to the disclosure.

In yet a further exemplary embodiment, the kit comprises at least three compartments or containers, where the first compartment or container comprises a base lightening composition according to the disclosure excluding one or more of the components described herein, for example excluding thermal control agents. In such an embodiment, a second compartment or container comprises the excluded component(s), for example comprises one or more thermal control agents, and a third compartment or container comprises an additive composition according to the disclosure. In such an embodiment, the hair lightening composition prepared by mixing the contents of the first, second, and third compartments or containers would result in a hair lightening composition according to the disclosure just as if the thermal control agent(s) had been included in the base lightening composition. Optionally, a fourth compartment or container which comprises an oxidizing composition.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “citric acid and/or salts thereof” means “citric acid and salts thereof” as well as “citric acid or salts thereof.”

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−5% from the stated number, such as +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions in which the component is present, unless otherwise indicated.

The term “altering the color” or “color altering” as used herein refers to lifting or lightening the color of hair.

For purposes of the present disclosure, naturally-curly (loose-to corkscrew-curl), natural level 4 hair that has a ΔL value of about 40 or higher when treated with a hair lightening composition as described herein is considered to achieve “high” levels of lift or lightening, and naturally-curly (loose-to corkscrew-curl), natural level 4 hair that has a ΔL value of about 54 or higher when treated with a hair lightening composition as described herein is considered to achieve “very high” levels of lift or lightening, when measured in the CIE L*a*b*system using ColorShot MS (Newtone Technologies, software version 1.8.0).

As used herein, the terms “applying a composition onto keratin fibers” and “applying a composition onto hair” and variations of these phrases are intended to mean contacting the keratin fibers including hair, with at least one of the compositions of the disclosure, in any manner. It may also mean contacting the keratin fibers or hair in an effective amount of the composition.

As used herein, the term “salts” may include salts having a counter-ion such as an alkali metal, alkaline earth metal, or ammonium counterion. This list of counterions, however, is non-limiting. Salts also include a dissociated form of a compound, e.g. in an aqueous solution.

Unless otherwise specified for a particular component, as used herein the terms “substantially free” or “essentially free” mean the component may be added in amounts less than 2% by weight, based on the total weight of the composition in which it is present, such as less than 1.75%, less than 1.5%, less than 1.25%, less than 1%, less than 0.75%, less than 0.5%, less than 0.25%, less than 0.1%, less than 0.05%, or less than 0.01% of the specified component. Unless expressly stated otherwise, “substantially free” or “essentially free” means less than 2%. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The terms “free,” “substantially free,” and “essentially free” as used herein mean that the specific component is not added to the composition but may still be present as an additive in small amounts of a raw material that is included in the composition.

As used herein, the term “surfactants,” as well as any specifically identified surfactants, includes salts of the surfactants even if not explicitly stated.

As used herein, the term “treat” (and its grammatical variations) refers to the application of the compositions of the present disclosure onto the surface of the specified keratin material, such as hair growing from the scalp.

As used herein, the phrase “at or near the time of use” is meant to indicate that the step of mixing or combining components or compositions is performed within a short time before the mixture is applied to the keratin fibers to be treated. For example, the consumer or professional colorist may perform the mixing step within an hour or less of the mixture being applied to the hair, such as 45 minutes or less, 30 minutes or less, or 15 minutes or less.

As used herein, the terms “mixed with,” “added to,” and the like are meant to denote a combining of two or more components or compositions, and should not be limited to a specific method or order of combining.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or description that the steps are to be limited to a specific order, it is no way intended that any particular order be inferred.

### EXAMPLES

The following examples are intended to be non-limiting and explanatory in nature only. In the Examples, amounts in each composition are expressed in percentage by weight (wt %) of active materials, unless otherwise defined, relative to the total weight of the composition.

### Example 1-Base Lightening, Additive, and Oxidizing Compositions

Base lightening compositions B1-B6 shown in Table 1-1 were prepared.

TABLE 1-1
Base Lightening Compositions

| | B1 | B2 | B3 | B4 | B5 | B6 |
|---|---|---|---|---|---|---|
| AMMONIUM | 12.80 | 13.35 | 12.80 | 13.03 | 11.60 | 11.61 |
| PERSULFATE | | | | | | |
| POTASSIUM | 29.35 | 24.15 | 29.35 | 23.60 | 17.00 | 21.01 |
| PERSULFATE | | | | | | |
| MINERAL OIL | 2.21 | 2.30 | 2.21 | 2.25 | 4.60 | 2.00 |
| SODIUM STEARATE | 4.17 | 4.35 | 4.17 | 4.25 | 4.60 | 3.78 |
| SODIUM SILICATE | 24.67 | 30.20 | 24.67 | 29.55 | 32.00 | 16.72 |
| SODIUM | 6.22 | 6.50 | 6.22 | 6.36 | 2.00 | 5.66 |
| METASILICATE | | | | | | |
| MAGNESIUM | 9.93 | 10.35 | 9.93 | 10.11 | 4.40 | 9.00 |
| CARBONATE | | | | | | |
| HYDROXIDE | | | | | | |
| HYDROXYETHYL- | 0.77 | 0.80 | 0.77 | 0.79 | 2.6 | 0.70 |
| CELLULOSE | | | | | | |
| ACRYLATES/ | 0.77 | 0.80 | 0.77 | 0.79 | 1.8 | 0.70 |
| C10-30 ALKYL | | | | | | |
| ACRYLATE | | | | | | |
| CROSSPOLYMER | | | | | | |
| SODIUM CITRATE | 6.23 | 4.20 | 4.02 | 4.10 | 3.4 | 3.65 |
| GLYCINE | | | 2.21 | 2.25 | 2.6 | 2.00 |
| DISODIUM EDTA | 1.10 | 1.15 | 1.10 | 1.12 | 1.00 | 1.00 |
| ULTRAMARINES + | 0.68 | 0.70 | 0.68 | 0.68 | 0.40 | 0.61 |
| KAOLIN | | | | | | |
| SODIUM LAURYL | 1.10 | 1.15 | 1.10 | 1.12 | 1.00 | 1.00 |
| SULFATE | | | | | | |
| β-CYCLODEXTRIN | | | | | | 0.18 |
| GLYCERIN | | | | | | 0.10 |
| HYDROXYPROPYL | | | | | | 0.11 |
| GUAR HYDROXY- | | | | | | |
| PROPYLTRIMONIUM | | | | | | |
| CHLORIDE | | | | | | |
| WATER | | | | | | QS to 100 |
| TOTAL PERSULFATES | 42.15 | 37.50 | 42.15 | 36.63 | 28.60 | 32.62 |

Base lightening composition B7 shown in Table 1-2 was prepared.

TABLE 1-2
Base Lightening Composition

| | B7 |
|---|---|
| AMMONIUM PERSULFATE | 11.60 |
| POTASSIUM PERSULFATE | 41.60 |
| MINERAL OIL | 2.00 |
| SODIUM STEARATE | 3.78 |
| SODIUM SILICATE | 16.72 |
| SODIUM METASILICATE | 5.64 |
| MAGNESIUM CARBONATE HYDROXIDE | 9.00 |
| HYDROXYETHYLCELLULOSE | 0.70 |
| ACRYLATES/C10-30 ALKYL ACRYLATE CROSSPOLYMER | 0.70 |
| GLYCINE | 2.00 |
| CITRIC ACID | 3.64 |
| DISODIUM EDTA | 1.00 |
| ULTRAMARINES + KAOLIN | 0.62 |
| SODIUM LAURYL SULFATE | 1.00 |
| TOTAL PERSULFATES | 53.20 |

Exemplary additive compositions A1-A4 shown in Table 1-3 were prepared.

TABLE 1-3
Additive Compositions

| | A1 | A2 | A3 | A4 |
|---|---|---|---|---|
| CITRIC ACID | 2.50 | 3.70 | 2.50 | 2.50 |
| β-CYCLODEXTRIN | 1.75 | 2.60 | 1.75 | 1.50 |
| GLYCERIN | 1.00 | 1.00 | 1.0 | 1.00 |
| HYDROXYPROPYL GUAR | 1.07 | 1.07 | 1.07 | |
| HYDROXYPROPYL- | | | | |
| TRIMONIUM | | | | |
| CHLORIDE | | | | |
| XANTHAN GUM | | | | 1.00 |
| SODIUM HYDROXIDE | | | QS to | |
| | | | pH 3.5 | |
| WATER | QS | QS | | QS |
| | to 100 | to 100 | | to 100 |

Compositions A1-A4 were prepared by first preparing a premixture of citric acid and cyclodextrin in deionized water and heating the mixture in a water bath at 80° C. until the citric acid and cyclodextrin dissolved and the mixture formed a viscous liquid, then cooling down the mixture to room temperature and adjusting the pH of the premixture as needed to reach pH of about 3.5 to 4, then adding the polysaccharide thickener and glycerin and mixing the combination using a vortex stirrer (IKA® EUROSTAR Power Control from IKA Werke) for about an hour to ensure that a homogeneous gel was obtained.

Oxidizing compositions (developers) D1-D2 shown in Table 1-4 were used.

TABLE 1-4
Oxidizing Compositions

| | D1 | D2 |
|---|---|---|
| TRIDECETH-2 CARBOXAMIDE MEA | 0.85 | |
| TETRASODIUM ETIDRONATE | 0.20 | 0.12 |
| SODIUM SALICYLATE | 0.04 | 0.05 |
| GLYCERIN | 0.50 | |
| CETEARYL ALCOHOL | 2.28 | 3.15 |
| CETEARETH-25 | 0.57 | |
| HYDROGEN PEROXIDE | 12.00 | 9.00 |
| TETRASODIUM PYROPHOSPHATE | 0.04 | 0.05 |
| PEG-40 HYDROGENATED CASTOR OIL | | 0.90 |
| SODIUM CETEARYL SULFATE | | 0.45 |
| MINERAL OIL | | 17.00 |
| WATER | QS to 100 | QS to 100 |

### Example 2-Comparison of Properties of Treated Hair

In order to evaluate the integrity of hair lightened with hair lightening compositions according to the disclosure, and the lightening efficacy of the compositions, comparative studies were conducted.

### Evaluation Methods

The methods used in the studies to evaluate the integrity of the treated hair fibers include break stress and elastic modulus evaluation, differential scanning calorimetry (DSC), and cyclic fatigue testing. Curl definition retention and lightening of the treated hair were also evaluated.

### Fiber Integrity Testing

Evaluation of break stress and elastic modulus was carried out using a Miniature Tensile Tester (MTT 688 from Dia-Stron Ltd) and a Fibre Dimensional Analysis System (FDAS) 770). Elastic modulus represents a measure of the hair's spring-like structure (elasticity). Elastic modulus provides fiber deformation information, so fiber with higher elastic modulus is more resistant to deformation. Break stress represents the force/area needed to break the hair fiber. A higher break stress represents a stronger hair fiber. Overall, the higher the break stress and/or the elastic modulus, the better the fiber integrity.

For each swatch, fifty (n=50) fibers per hair sample were tested at 80% relative humidity and room temperature. Each hair swatch sample was soaked in deionized (DI) water for about two (2) hours, and subsequently subjected to MTT testing. Tensile measurement was performed while the hair fibers were wet, with 2 grams of force at a traction rate of about 40 mm/min. The measured results of the hair break stress and elastic modulus were statistically analyzed using UVWin software vision 4.2.5.0, where the statistical significance level was set at p<0.05.

### Differential Scanning Calorimetry (DSC)

Differential Scanning calorimetry (DSC) measures the temperatures and heat flow associated with the thermal transition in a material. It is useful for investigating the structural characteristics of hair fibers. Keratin undergoes detectable transformations at various temperatures. Changes in these transformation temperatures can be used to estimate how a particular hair treatment that may impact hair fibers. In the instant case, DSC was used to measure denaturation temperature (Ta). Denaturation temperature (Ta) has been used as a representation of the thermal stability of hair fibers, which is influenced, at least in part, by the cross-link density of the matrix (intermediate filament associated proteins, IFAP). Generally, even a slight increase in Ta shows improved hair strength/integrity.

Denaturation temperatures of the treated hair swatches were measured using a DSC 2500 Discovery series from TA Instruments. To prepare hair samples for analysis, 7-8 gram of each tested hair swatch was cut into particles <0.5 mm. The hair particles of each hair swatch were placed in a high-volume DSC pan chamber. About 50 μL of deionized water was added into the pan and the hair particles were soaked in the water. The pan chamber was then sealed using a crimping dime. For each treated hair swatch samples, five pans were prepared (n=5). DSC pans were equilibrated at room temperature for overnight. About 24 hours after treating the hair, the DSC pans were run on the DSC equipment starting from 40° C. and ramping by 10° C./min to 180° C. The Ta value was measured in degree Celsius, which represents the cross-linking density of the hair matrix. Trios' software was used to statistically analyze the denaturation temperature values measured by the DSC equipment.

### Cyclic Fatique Tensile Testing (CFTT)

Cyclic fatigue testing is typically used to simulate the repeated tensile stress that occurs while combing and brushing. Cyclic fatigue data and survival probability indicate the degree of both internal fiber flaws and external cuticle layer rupture. Cyclic fatigue testing provides a unique instrumental tool to demonstrate the progressive levels of impaired hair fibers. Fibers with relatively larger flaws will break at lower numbers of cycles of applied stress than fibers with minor defects.

Fiber durability testing (cycles to break (CTB)) was carried out using a Cyclic Fatigue Tensile Tester (CFTT) 801 from Dia-Stron Ltd. The hair sample size for each swatch was about fifty (n=50) fibers per sample. The experiment was carried out on dry hair fibers at room temperature, 45% relative humidity, and using a constant stress over a specified area.

### Evaluation of Frizz Control

Hair frizz control was measurement was carried out using an instrument system called HEAVEN (Hair Evaluation for Automated Volume Estimation) (Newton). The system took pictures of hair swatches at a desired time point. The software acquired images of the hair swatches loaded inside the system equipped with a humidity and temperature control unit.

The treated hair swatches were placed in the HEAVEN system having 80% relative humidity at room temperature for 24 hours, during which time the software acquired images of the swatches every hour.

### Evaluation of Curl Retention

RUMBA is a polarization imaging system that takes successive images of a hair sample illuminated with infrared LED and under several polarization states, using a rotating polarizer. The images give a modulated intensity on each pixel. The modulation is caused by the birefringence of the hair fiber and contains the angle signature that can be extracted with the system's software to build an orientation image of the sample. The orientation data can be mapped onto a 2D image to visualize the whole sample. From this orientation data, a coefficient that relates to straightness of the hair can be defined—the straighter the hair fibers in the swatch, the closer to a single angle value the distribution of the orientation will be. The standard deviation of such a distribution is therefore inversely proportional to the perception of straightness. The same process is done for the alignment, where the alignment of a hair swatch is defined as the hair fibers being “locally straight.” Thus, a straightness coefficient is defined on a small scale of the 2D mapping of the orientation, which allows for a straightness coefficient to be obtained on every pixel of the image.

After the hair swatches were removed from the HEAVEN system, curl definitions of the hair swatches were analyzed using RUMBA (Orientation measurement for straightness & Alignment of hair fiber) from Bossa Nova Technologies, LLC and/or with photographic images. Each individual hair swatch was placed in the RUMBA and images were acquired for the hair swatches exposed to a condition of 80% relative humidity at room temperature for 24 hours.

### Evaluation of Hair Lightening

The change in the color of hair was evaluated with the CIE L*a*b*system using ColorShot MS (Newtone Technologies, software version 1.8.0), where the change is determined by evaluating the color of the hair after treatment (L*₂, a*₂, b*₂) compared to the color of the hair before treatment (L*₁, a*₁, b*₁). In this system, L*indicates lightness, a*indicates red/green color, and b*indicates blue/yellow color. The change in lightness (ΔL) of the hair, which is used to evaluate the level of lift achieved, is calculated with equation (X¹), as follows:

$$Δ L = ( {L}_{2}^{⋆} ) - ( {L}_{1}^{⋆} ) . \qquad ( {X}^{{}_{1}} )$$

The change in color (ΔE, 1976) of the hair is calculated with equation (X²), as follows:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} . \qquad ( {X}^{{}_{2}} )$$

### Example 2A

Just before use, 89 grams of base lightening composition B4 was mixed with 11 grams of additive composition A1 to form base lightening mixture B4′ according to the disclosure. Base lightening mixture B4′ was then mixed with 150 grams of developer D1 to produce hair lightening composition 2A according to the disclosure. Hair lightening composition 2A was applied to a swatch of curly (curl type 4) natural level 4 hair (swatch S1) at a rate of about 10 grams per gram of hair, covered with foil, and left to process at 33° C. for 40 minutes. The foil was removed and the swatch was rinsed with water, shampooed with a commercially available shampoo, rinsed again, and left to air dry overnight at room temperature. The following day, the swatch was treated with a second application of the same composition as used in the first application. The swatch was again covered with foil and left to process at 33° C. for 40 minutes, and then rinsed, shampooed with a commercially available shampoo, and rinsed again.

Just before use, 100 grams of base lightening composition B7 was mixed with 150 grams of developer D2 to produce comparative hair lightening composition C1. Comparative hair lightening composition C1 was applied to a swatch of curly (curl type 4) natural level 4 hair (swatch S2) at a rate of about 10 grams per gram of hair, covered with foil, and left to process at 33° C. for 50 minutes. The foil was removed and the swatch was rinsed with water, shampooed with a commercially available shampoo, rinsed again, and left to air dry overnight at room temperature. The following day, the swatch was treated with a second application of the same composition as used in the first application. The swatch was again covered with foil and left to process at 33° C. for 40 minutes, and then rinsed, shampooed with a commercially available shampoo, and rinsed again.

Subsequently, MTT, DSC, CFTT, Heaven, Rumba, and ColorShot data were collected for swatches S1-S2 according to the above protocols. The results are shown in Table 2-1.

TABLE 2-1

| METHOD | | 2A (S1) | C1 (S2) |
|---|---|---|---|
| MTT - Elastic Modulus | (MPa) mean ± SD | 928.4 ± 242.1 | 837.0 ± 218.9 |
| MTT - Break Stress | (MPa) mean ± SD | 112.7 ± 18.6 | 85.7 ± 29.1 |
| CFTT | Cycles to break | 867 | 433 |
| DSC - T_{d} | (° C.) mean ± SD | 132.9 ± 1.2 | 131.0 ± 0.5 |
| DSC - ΔH_{d} | (J/g) mean ± SD | 14.7 ± 3.8 | 11.7 ± 0.8 |
| Heaven | Frizz Area (cm²) | 24 | 46.2 |
| Rumba | Avg. alignment coef. (1/°) | 16.5 | 10.5 |
| ColorShot | ΔE | 75 | 75.6 |
| | ΔL | 59.4 | 60.8 |

As a difference in ΔL of 2 or less would not be observable to the naked eye, the ColorShot data in Table 2-1 demonstrate that the lightening efficacy of compositions 2A and C1, at the specified processing parameters, was equivalent (meaning the same level of lift was achieved). However, all of the integrity data demonstrates that swatch S1 had significantly less damage from the lightening process than swatch S2, which indicates that the hair fibers treated with hair lightening compositions according to the disclosure are more durable and resistant to breakage.

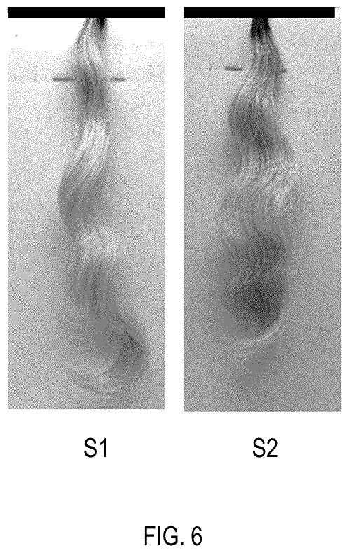

In addition, as FIG. 6 shows, swatch S1 had less frizz and volume and was more aligned compared to swatch S2, meaning that swatch S1 treated with hair lightening composition 2A according to the disclosure retained the curl better and was less frizzy than swatch S2.

### Example 2B

Just before use, 100 grams of base lightening composition B5 was mixed with 11 grams of additive composition A2 to form base lightening mixture B5′ according to the disclosure. Base lightening mixture B5′ was then mixed with 200 grams of developer D1 to produce hair lightening composition 2B according to the disclosure. Hair lightening composition 2B was applied to three swatches of curly (curl type 4) natural level 4 hair (swatches S3-S5) at a rate of about 10 grams per gram of hair, covered with foil, and left to process at 33° C. for 45 minutes. The foil was removed and the swatches were rinsed with water, shampooed with a commercially available shampoo, rinsed again, and left to air dry overnight at room temperature. The following day, the swatches were treated with a second application of the same composition as used in the first application. The swatches were again covered with foil and left to process at 33° C. for 45 minutes, and then rinsed, shampooed with a commercial shampoo, and rinsed again.

Just before use, 100 grams of base lightening composition B5 was mixed with 200 grams of developer D1 to produce comparative hair lightening composition C2. Comparative hair lightening composition C2 was applied to three swatches of curly (curl type 4) natural level 4 hair (swatches S6-S8) at a rate of about 10 grams per gram of hair, covered with foil, and left to process at 33° C. for 45 minutes. The foil was removed and the swatches were rinsed with water, shampooed with a commercially available shampoo, rinsed again, and left to air dry overnight at room temperature. The following day, the swatches were treated with a second application of the same composition as used in the first application. The swatches were again covered with foil and left to process at 33° C. for 45 minutes, and then rinsed, shampooed with a commercial shampoo, and rinsed again.

Subsequently, MTT, DSC, CFTT, Heaven, Rumba, and ColorShot data were collected for swatches S3-S8 according to the above protocols. The results are shown in Table 2-2, where the number reported for composition 2B is the average for swatches S3-S5, and the number reported for composition C2 is the average for swatches S6-S8.

TABLE 2-2

| METHOD | | 2B (S3-S5) | C2 (S6-S8) |
|---|---|---|---|
| MTT - Elastic Modulus | (MPa) mean ± SD | 1049.4 ± 715.5 | 888.7 ± 333.3 |
| MTT - Break Stress | (MPa) mean ± SD | 130.8 ± 20.1 | 112.8 ± 25.4 |
| CFTT | Cycles to break | 2822 | 927 |
| DSC - T_{d} | (° C.) mean ± SD | 133.9 ± 0.2 | 132.3 ± 0.4 |
| DSC - ΔH_{d} | (J/g) mean ± SD | 14.2 ± 0.4 | 14 ± 0.63 |
| Heaven | Frizz Area (cm²) | 50.1 ± 3.48 | 68 ± 11.7 |
| Rumba | Avg. alignment coef. (1/°) | 9.9 ± 0.17 | 7.7 ± 0.33 |
| ColorShot | ΔE | 71.2 | 72.9 |
| | ΔL | 55.2 | 56.1 |

As a difference in ΔL of 2 or less would not be observable to the naked eye, the ColorShot data in Table 2-2 demonstrate that the lightening efficacy of compositions 2B and C₂, at the specified processing parameters, was equivalent. However, all of the integrity data demonstrates that swatches S3-S5 had significantly less damage from the lightening process than swatches S6-S8, which indicates that the hair fibers treated with hair lightening compositions according to the disclosure are more durable and resistant to breakage.

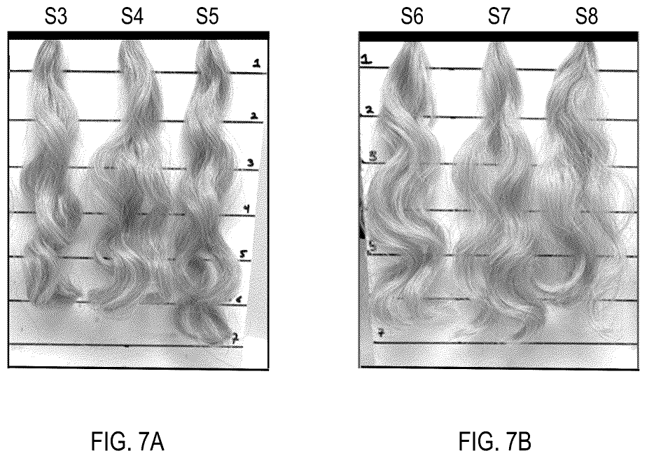

In addition, as FIGS. 7A-7B show, swatches S3-S5 had tighter curls, less frizz and volume, and were more aligned compared to swatches S6-S8, meaning that swatches treated with hair lightening composition 2B according to the disclosure retained the curl better and were less frizzy than swatches treated with comparative hair lightening compositions.

This Example therefore demonstrates that hair lightened with a lightening composition to which an additive composition has been added has surprisingly better integrity and cosmetic properties, compared to an otherwise identical hair lightening composition.

### Example 2C

Just before use, 87 grams of base lightening composition B2 was mixed with 3 grams of water and 10 grams of additive composition A1 to form base lightening mixture B2′ according to the disclosure. Base lightening mixture B2′ was then mixed with 150 grams of developer D1 to produce hair lightening composition 2C according to the disclosure. Hair lightening composition 2C was applied to a swatch of curly (curl type 4) natural level 4 hair (swatch S9) at a rate of about 10 grams per gram of hair, covered with foil, and left to process at 33° C. for 35 minutes. The swatch was then rinsed, shampooed with a commercial shampoo, and rinsed again.

Just before use, 100 grams of base lightening composition B7 was mixed with 150 grams of developer D2 to produce comparative hair lightening composition C3. Hair lightening composition C3 was applied to a swatch of curly (curl type 4) natural level 4 hair (swatch S10) at a rate of about 10 grams per gram of hair, covered with foil, and left to process at 33° C. for 50 minutes. The swatch was then rinsed, shampooed with a commercial shampoo, and rinsed again.

Just before use, 87 grams of base lightening composition B2 was mixed with 13 grams of water to form comparative base lightening mixture CB2. Comparative base lightening mixture CB2 was mixed with 150 grams of developer D1, to produce comparative hair lightening composition C4. Comparative hair lightening composition C4 was applied to a swatch of curly (curl type 4) natural level 4 hair (swatch S11) at a rate of about 10 grams per gram of hair, covered with foil, and left to process at 33° C. for 35 minutes. The swatch was then rinsed, shampooed with a commercial shampoo, and rinsed again.

Just before use, 100 grams of base lightening composition B6 was mixed with 150 grams of developer D1, to produce hair lightening composition 2D according to the disclosure. Hair lightening composition 2D was applied to a swatch of curly (curl type 4) natural level 4 hair (swatch S12) at a rate of about 10 grams per gram of hair, covered with foil, and left to process at 33° C. for 35 minutes. The swatch was then rinsed, shampooed with a commercial shampoo, and rinsed again.

Subsequently, MTT, DSC, CFTT, Heaven, Rumba, and ColorShot data were collected for swatches S9-S12 according to the above protocols. The results are shown in Table 2-3.

TABLE 2-3

| METHOD | | 2C (S9) | C3 (S10) | C4 (S11) | 2D (S12) |
|---|---|---|---|---|---|
| MTT - | (MPa) | 1482.7 ± 173 | 1111.6 ± 179.8 | 1380.3 ± 168.4 | 1381.5 ± 146.1 |
| Elastic | mean ± SD | | | | |
| Modulus | | | | | |
| CFTT | Cycles to | 5072 | 4402 | 1861 | 6165 |
| | break | | | | |
| Rumba | Avg. | 12.1 | 5.9 | 6.4 | 8.2 |
| | alignment | | | | |
| | coef. (1/°) | | | | |
| Heaven | Frizz Area | 25 | 80.2 | 79 | 47 |
| | (cm²) | | | | |
| DSC - T_{d} | (° C.) mean ± | 142.6 ± 0.6 | 144.5 ± 1.0 | 141.0 ± 0.9 | 142.0 ± 0.3 |
| | SD | | | | |
| ColorShot | ΔE | 69.7 | 65.7 | 66.9 | 65.5 |
| | ΔL | 44.5 | 43.9 | 44.3 | 42.6 |

As a difference in ΔL of 2 or less would not be observable to the naked eye, the ColorShot data in Table 2-3 demonstrate that the lightening effect of compositions 2C-2D and C3-C4 are equivalent under the specified processing parameters.

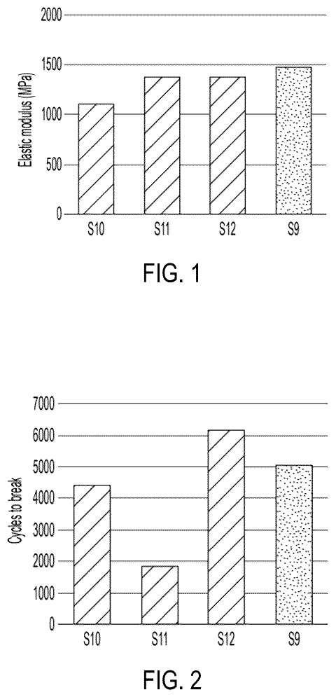

However, the fiber integrity data in Table 2-3 show, and also shown graphically in FIGS. 1-2, swatches S9 and S12 treated with hair lightening compositions according to the disclosure demonstrate improved strength and durability compared to that of swatches S10-S11. Swatches S9 and S12 had the highest elasticity and Ta values, respectively. Swatch S12 had the highest value for CFTT, followed by swatch 9.

Although the measured elasticity for swatch S11 was only slightly below that of swatch S12, the CFTT and Ta values for swatch S11 were significantly below those of both swatches S9 and S12. Taken as a whole, these data demonstrate that hair lightening compositions 2C and 2D according to the disclosure surprisingly provide improved integrity, durability, and protein cross-linked density to hair treated under comparable conditions with comparable hair lightening compositions not according to the disclosure.

While it was observed that swatch S10 had the highest Ta value, it is believed that this is due to the use of a lower strength (30V) developer in composition C3, which would explain the lower ΔE value even though the processing time was longer. It is expected that if an equivalent strength of developer (40V) was used in composition C3, even if the processing time was reduced to 35 minutes the ΔE value and damage would both increase, leading to a decrease in the Ta value and corresponding increase in the MTT and CFTT values.

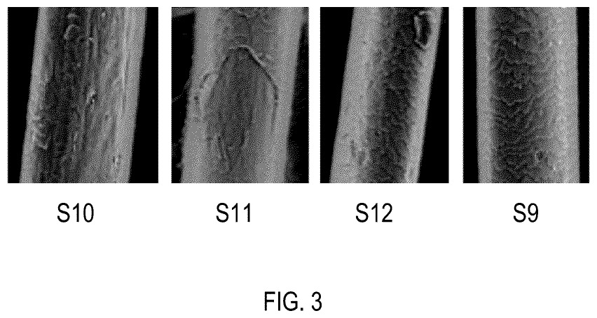

Scanning Electron Microscope (SEM) images of fibers from each of swatches S9-S12 were taken to analyze the surface of the hair fibers. Representative images for each of S9-S12 are seen in FIG. 3. As FIG. 3 shows, swatches S10-S11 show significant defects on the hair cuticle, which is consistent with the MTT and CTFF data in Table 2-3. While swatch S12 showed some minor cuticle defects, the image shows that the scales are intact and aligned. In addition, swatch S9 had no visible defects and had scales that are intact and aligned. This data further demonstrates that hair lightening compositions 2C-2D according to the disclosure provided increased protection to the hair fibers during the hair lightening process.

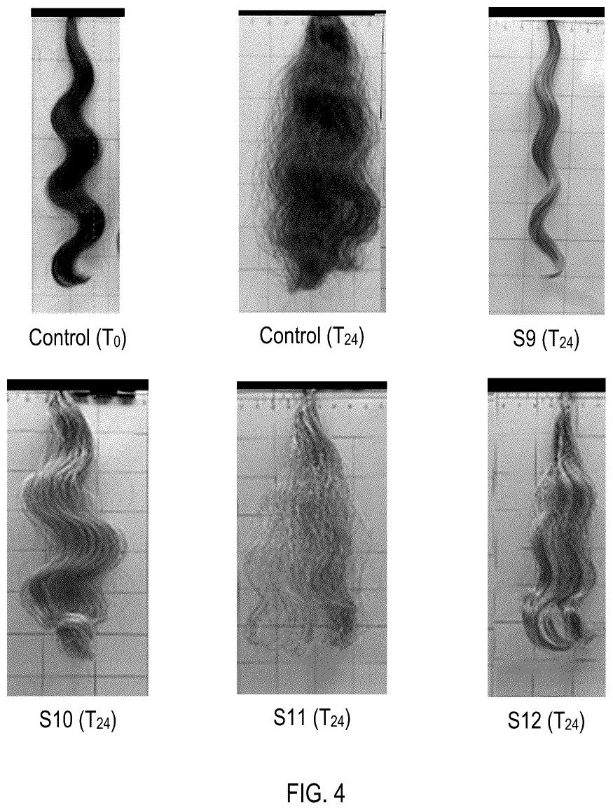

An untreated swatch (control) and swatches S9-S12 were exposed to 80% relative humidity at room temperature for 24 hours. FIG. 4 shows images of the untreated swatch before (T₀) and after (T₂₄) exposure to high humidity conditions, and of swatches S9-S12 after exposure to high humidity conditions. As can be seen in FIG. 4, swatches S9 and S12, treated with hair lightening compositions according to the disclosure, had significantly less frizz and volume and better curl retention than swatches S10-S11, even after exposure to conditions of high humidity.

These data therefore also demonstrate that hair lightening compositions 2C-2D according to the disclosure provided improved cosmetic benefits to the hair.

### Example 2D

Just before use, 87 grams of base lightening composition B2 was mixed with 3 grams of water and 10 grams of additive composition A3 to form base lightening mixture B2″ according to the disclosure. Base lightening mixture B2″ was then mixed with 150 grams of developer D1 to produce hair lightening composition 2E according to the disclosure. Hair lightening composition 2E was applied to a swatch of straight natural level 4 hair (swatch S13) at a rate of about 10 grams per gram of hair, covered with foil, and left to process at 33° C. for 35 minutes. The swatch was then rinsed, shampooed with a commercial shampoo, rinsed again, and allowed to air dry.

Just before use, 87 grams of base lightening composition B2 was mixed with 3 grams of water to form comparative base lightening mixture CB2′. Comparative base lightening mixture CB2′ was mixed with 150 grams of developer D1 to produce comparative hair lightening composition C5. Comparative hair lightening composition C5 was applied to a swatch of straight natural level 4 hair (swatch S14) at a rate of about 10 grams per gram of hair, covered with foil, and left to process at 33° C. for 35 minutes. The swatch was then rinsed, shampooed with a commercial shampoo, rinsed again, and allowed to air dry.

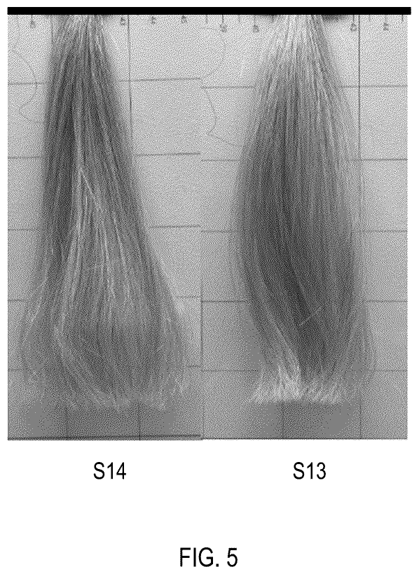

FIG. 5 shows images of swatches S13 and S14 once dried. As can be seen, swatch S13 has less frizz, the fibers are more aligned, the ends are more sealed, and the overall appearance is healthier than swatch S14. In addition, swatch S13 was considered to be softer and smoother to the touch than swatch S14.

The results in the Examples demonstrate that hair treated with hair lightening compositions according to the disclosure has improved integrity and curl retention, and less frizz, compared to hair treated with traditional hair lightening compositions, resulting in smoother, healthier looking and feeling hair compared to hair treated with traditional hair lightening compositions. Based on these Examples, it is expected that other combinations of additive compositions according to the disclosure, base lightening compositions, and developer compositions would provide similar frizz control, fiber alignment, smoothness, and lightening results.

1. A hair lightening composition prepared by mixing:
(1) a hair base lightening composition comprising:
(a) at least two persulfate compounds comprising:
(i) ammonium persulfate; and
(ii) at least one additional persulfate compound;
(b) at least one silicate compound; and
(c) optionally at least one metasilicate compound,
(2) an additive composition comprising:
(a) at least one carboxylic acid or salt thereof;
(b) at least one cyclodextrin or derivative thereof;
(c) at least one polysaccharide thickening agent;
(d) at least one polyol; and
(e) water, and
(3) an oxidizing agent,
wherein the total amount of persulfate compounds in the hair base lightening composition ranges from about 10% to not greater than 50% by weight, relative to the total weight of the hair base lightening composition, and
wherein the weight ratio of (1) (a) (i):(1) (a) (ii) in the hair base lightening composition is less than 1.

2. The hair lightening composition according to claim 1, wherein the total amount of persulfate compounds ranges from about 25% to about 45% by weight, relative to the total weight of the hair base lightening composition.

3. The hair lightening composition according to claim 1, wherein the weight ratio of (1) (a) (i):(1) (a) (ii) in the hair base lightening composition ranges from about 0.1 to about 0.9.

4. The hair lightening composition according to claim 1, wherein the total amount of silicate compounds ranges from about 5% to about 50% by weight, relative to the total weight of the hair base lightening composition.

5. The hair lightening composition according to claim 1, wherein the mole ratio of (2) (a):(2) (b) in the additive composition ranges from about 0.5:1 to about 25:3, and/or the weight ratio of (2) (a):(2) (b) in the additive composition ranges from about 1:3 to about 6:1.

6. The hair lightening composition according to claim 1, wherein the at least one carboxylic acid or salt thereof is chosen from mono-carboxylic acids, di-carboxylic acids, tri-carboxylic acids, salts thereof, or mixtures of two or more thereof.

7. The hair lightening composition according to claim 1, wherein the total amount of carboxylic acids and salts thereof ranges from about 1% to about 20% by weight, relative to the total weight of the additive composition.

8. The hair lightening composition according to claim 1, wherein the at least one cyclodextrin or derivative thereof is chosen from compounds of the following formula or mixtures of two or more thereof: 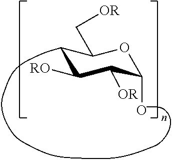
wherein:
R is chosen from H, CH₃, or a hydroxypropyl group; and
n ranges from 6-8.

9. The hair lightening composition according to claim 1, wherein the total amount of cyclodextrins and derivatives thereof ranges from about 0.01% to about 20% by weight, relative to the total weight of the additive composition.

10. The hair lightening composition according to claim 1, wherein the total amount of polysaccharide thickening agents ranges from 0.05% to about 5% by weight, relative to the total weight of the additive composition.

11. The hair lightening composition according to claim 1, wherein the total amount of polyols is at least 0.5% by weight, relative to the total weight of the additive composition.

12. The hair lightening composition according to claim 1, prepared by mixing:
(1) a hair base lightening composition comprising:
(a) at least two persulfate compounds comprising:
(i) ammonium persulfate; and
(ii) at least one additional persulfate compound;
(b) at least one silicate compound; and
(c) optionally at least one metasilicate compound,
(2) an additive composition comprising:
(a) at least one carboxylic acid chosen from citric acid, lactic acid, malic acid, maleic acid, tartaric acid, glycolic acid, phytic acid, salts thereof, or mixtures of two or more thereof;
(b) at least one cyclodextrin chosen from α-cyclodextrin, ß-cyclodextrin, γ-cyclodextrin, methyl derivatives of α-cyclodextrin, methyl derivatives of ß-cyclodextrin, methyl derivatives of γ-cyclodextrin, hydroxypropyl l derivatives of α-cyclodextrin, hydroxypropyl derivatives of ß-cyclodextrin, hydroxypropyl derivatives of γ-cyclodextrin, or mixtures of two or more thereof;
(c) at least one polysaccharide thickening agent chosen from gum arabic, tragacanth gum, karaya gum, guar gum, gellan gum, tara gum, locust bean gum, tamarind gum, xanthan gum, dehydroxanthan gum, Seneca gum, sclerotium gum, gellan gum, or mixtures of two or more thereof;
(d) at least one polyol chosen from ethylene glycol, propylene glycol, butylene glycol, hexylene glycol, pentylene glycol, diethylene glycol, dipropylene glycol, 1,3 propanediol, glycerin, polyethylene glycols, or mixtures of two or more thereof; and
(e) water, and
(3) an oxidizing agent,
wherein the total amount of persulfate compounds in the hair base lightening composition ranges from about 10% to not greater than 50% by weight, relative to the total weight of the hair base lightening composition,
wherein the weight ratio of (1) (a) (i):(1) (a) (ii) in the hair base lightening composition is less than 1, and
wherein the mole ratio of (2) (a):(2) (b) in the additive composition ranges from about 0.5:1 to about 25:3, and/or the weight ratio of (2) (a):(2) (b) in the additive composition ranges from about 1:3 to about 6:1.

13. A method of treating hair comprising:
preparing a hair lightening composition by mixing:
(1) a hair base lightening composition comprising:
(a) at least two persulfate compounds comprising:
(i) ammonium persulfate, and
(ii) at least one additional persulfate compound;
(b) at least one silicate compound; and
(c) optionally at least one metasilicate compound,
wherein the total amount of persulfate compounds in the hair base lightening composition ranges from about 10% to not greater than 50% by weight, relative to the total weight of the hair base lightening composition, and
wherein the weight ratio of (1) (a) (i):(1) (a) (ii) in the hair base lightening composition is less than 1;
(2) an additive composition comprising,
(a) at least one carboxylic acid or salt thereof;
(b) at least one cyclodextrin or derivative thereof;
(c) at least one polysaccharide thickening agent;
(d) at least one polyol; and
(e) water; and
(3) at least one oxidizing agent, and
applying the hair lightening composition to the hair.

14. A hair base lightening mixture prepared by mixing:
(1) a hair base lightening composition comprising:
(a) at least two persulfate compounds comprising:
(i) ammonium persulfate; and
(ii) at least one additional persulfate compound;
(b) at least one silicate compound;
(c) optionally at least one metasilicate compound, and
(2) an additive composition comprising:
(a) at least one carboxylic acid or salt thereof;
(b) at least one cyclodextrin or derivative thereof;
(c) at least one polysaccharide thickening agent;
(d) at least one polyol; and
(e) water,
wherein the total amount of persulfate compounds in the hair base lightening composition ranges from about 10% to not greater than 50% by weight, relative to the total weight of the hair base lightening composition, and
wherein the weight ratio of (1) (a) (i):(1) (a) (ii) in the hair base lightening composition is less than 1.

15. The hair base lightening mixture according to claim 14, wherein the mole ratio of (2) (a):(2) (b) in the additive composition ranges from about 0.5:1 to about 25:3, and/or the weight ratio of (2) (a):(2) (b) in the additive composition ranges from about 1:3 to about 6:1.

16. The hair base lightening mixture according to claim 14, wherein:
the at least one carboxylic acid or salt thereof is chosen from mono-carboxylic acids, di-carboxylic acids, tri-carboxylic acids, salts thereof, or mixtures of two or more thereof, and/or
the at least one cyclodextrin or derivative thereof is chosen from compounds of the following formula or mixtures of two or more thereof: 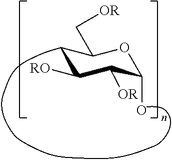
wherein:
R is chosen from H, CH₃, or a hydroxypropyl group; and
n ranges from 6-8.

17. The hair base lightening mixture according to claim 14, wherein the total amount of carboxylic acids and salts thereof ranges from about 1% to about 20% by weight, relative to the total weight of the additive composition.

18. The hair base lightening mixture according to claim 14, wherein the total amount of cyclodextrins and derivatives thereof ranges from about 0.01% to about 20% by weight, relative to the total weight of the additive composition.

19. The hair base lightening mixture according to claim 14, wherein the total amount of polysaccharide thickening agents ranges from 0.05% to about 5% by weight, relative to the total weight of the additive composition.

20. The hair base lightening mixture according to claim 14, wherein the total amount of polyols is at least 0.5% by weight, relative to the total weight of the additive composition.

21. A kit comprising:
(1) a first container or compartment comprising a hair base lightening composition comprising:
(a) at least two persulfate compounds comprising:
(i) ammonium persulfate; and
(ii) at least one additional persulfate compound;
(b) at least one silicate compound; and
(c) optionally at least one metasilicate compound,
(2) a second container or compartment comprising an additive composition comprising:
(a) at least one carboxylic acid or salt thereof;
(b) at least one cyclodextrin or derivative thereof;
(c) at least one polysaccharide thickening agent;
(d) at least one polyol; and
(e) water, and
(3) optionally, a third container or compartment comprising an oxidizing composition.
